(19) United States
(12) Patent Application Publication
(10) Pub. No.: US 2010/0099700 A1
(43) Pub. Date: Apr. 22, 2010

(54) HYDROGENATED PYRIDO (4,3-B) INDOLES FOR TREATING AMYOTROPHIC LATERAL SCLEROSIS (ALS)

(76) Inventor: David Hung, Redwood City, CA (US)

Correspondence Address: MORRISON & FOERSTER LLP, 755 PAGE MILL RD, PALO ALTO, CA, 94304-1018, US

(21) Appl. No.: 12/442,388

(22) PCT Filed: Sep. 20, 2007
(86) PCT No.: PCT/US07/20516

§ 371 Date: Dec. 28, 2009

Related U.S. Application Data
(60) Provisional application No. 60/846,139, filed on Sep. 20, 2006.

(51) Int. Cl. A61K 31/437; C07D 471/04

(52) U.S. Cl. 514/292; 546/85

(57) ABSTRACT

The invention provides methods for treating and/or preventing and/or slowing the onset and/or development of ALS using hydrogenated pyrido(4,3-b)indoles, such as dimebon.

### CROSS-REFERENCE TO RELATED APPLICATIONS

This application claims priority to U.S. Provisional Patent Application No. 60/846,139, filed Sep. 20, 2006, which is incorporated herein by reference in its entirety.

### STATEMENT OF RIGHTS TO INVENTIONS MADE UNDER FEDERALLY SPONSORED RESEARCH

Not applicable.

### TECHNICAL FIELD

The present invention relates to methods and compositions useful for treating, preventing and/or delaying the onset and/or development of amyotrophic lateral sclerosis (ALS) by administering a hydrogenated pyrido[4,3-b]indole, or a pharmaceutically acceptable salt thereof to an individual.

### BACKGROUND OF THE INVENTION

Neurodegenerative diseases are generally characterized by a degeneration of neurons in either the brain or the nervous system of an individual. These diseases can be debilitating, and the damage that they cause is often irreversible.

### Summary of Amyotrophic Lateral Sclerosis Pathology

Amyotrophic lateral sclerosis (ALS), also called Lou Gehrig's disease, is a universally fatal neurodegenerative condition in which patients progressively lose all motor function. ALS has both familial (5-10%) and sporadic forms. The familial forms (FALS) have now been linked to several distinct genetic loci. About 15-20% of familial cases are due to mutations in the gene encoding Cu/Zn superoxide dismutase 1 (SOD1). Given the clinical and epidemiological similarity between sporadic and FALS, an understanding of the familial disease may illuminate possible pathophysiological mechanisms in sporadic ALS.

ALS involves the attack of motor neurons in the cortex, brain stem and spinal cord. The progressive degeneration of these nerve cells often leads to their death. As motor neurons die, they lose the ability to stimulate muscle fibers, and consequently, the brain loses the ability to initiate and control muscle movement. In later stages of the disease, patients become totally paralyzed, yet retain their cognitive functioning.

Early symptoms of ALS include increasing muscle weakness, particularly in the arms and legs and in the muscles associated with speech, swallowing and breathing. Symptoms of weakness and muscle atrophy usually begin asymmetrically and distally in one limb, and then spread within the neuroaxis to involve contiguous groups of motor neurons. Symptoms can begin either in bulbar or limb muscles. Clinical signs of both lower and upper motor neuron involvement are required for a definitive diagnosis of ALS. Respiration is usually affected late in limb onset patients, but occasionally can be an early manifestation in patients with bulbar onset symptoms.

Unable to walk, speak or breathe on their own, ALS patients die within two to five years of diagnosis. The incidence of ALS increases substantially in the fifth decade of life. ALS affects approximately 30,000 Americans with nearly 8,000 deaths reported in the US each year. ALS remains one of the most devastating diseases, and advances in treatment are desperately needed.

### Summary of Amyotrophic Lateral Sclerosis Pathogenesis

Although a great deal is known about the pathology of ALS, little is known about the pathogenesis of the sporadic form and about the causative properties of mutant SOD protein in familial ALS. Many models have been speculated, including hypoxia, oxidative stress, protein aggregates, neurofilament and mitochondrial dysfunction, atypical poliovirus infection, intoxication by exogenous metal-toxins, autoimmune processes targeting motor neurons, cytoskeletal abnormalities, trophic factor deprivation and toxicity from excess excitation of the motor neuron by transmitters such as glutamate. The motor neuron death process in ALS may reflect a complex interplay between oxidative injury, excitotoxic stimulation of the motor neurons, and dysfunction of mitochondria and critical proteins such as neurofilaments.

### Role of Oxidative Injury in Pathogenesis of Amyotrophic Lateral Sclerosis

As noted above, genetic studies have established that in some cases of FALS the primary defects are mutations in the gene for cytosolic, copper-zinc superoxide dismutase (SOD1). More than thirty five different mutations in SOD1 have been reported exclusively in FALS. SOD1 is a metalloenzyme of about 153 amino acids that is expressed in all eukaryotic cells. It is one of a family of three SOD enzymes, which include manganese-dependent, mitochondrial SOD (SOD2) and copper/zinc extracellular SOD (SOD3). The primary function of the SOD1 enzyme is believed to be detoxification of the superoxide anion by conversion to hydrogen peroxide. Hydrogen peroxide is subsequently detoxified by glutathione peroxidase or catalase to form water. Superoxide is potentially toxic by itself, and also can produce the more toxic hydroxyl radical either through formation of hydrogen peroxide or by reaction with nitric oxide. Superoxide also interacts with nitric oxide and forms peroxynitrite anion which may be directly toxic to cells and also generates hydroxyl radicals. An important implication of these biochemical properties of SOD1 is that FALS may arise as a consequence of abnormalities of free radical homeostasis and resulting cellular oxidative stress. Given the similarities between sporadic and familial ALS, sporadic ALS may also be a free radical disease.

The effects of the FALS mutations on SOD1 function are not fully understood. Many FALS-associated SOD1 mutations reduce SOD1 activity in tissues such as the brain and erythrocytes. In vitro, the mutations appear generally to alter stability of the mutant molecule, shortening the half-lives of the mutant proteins without necessarily reducing the specific activity of the SOD1 molecule. Why these mutations cause neuronal cell death remains unclear. In chronic organotypic spinal cord cultures, partial reduction of activity of SOD1 by chronic application of SOD1 anti-sense oligonucleotides triggers apoptotic nerve cell death, including fulminant motor neuron death. The death process, in vitro, is reversed by agents which enhance anti-oxidant defenses.

However, some lines of evidence suggest that the disease arises not from loss of SOD1 function, but rather from an adverse or novel property of the mutant SOD1 molecule. Dominantly inherited diseases, like FALS, are thought to arise because a single mutant allele produces a mutant protein with a novel property that is, in some way, toxic to the cell. Several laboratories have now demonstrated that mice which over-express high levels of mutant SOD1 protein develop a lethal, denervating, paralytic disease that resembles ALS clinically and pathologically. These findings support the hypothesis that the primary effect of the SOD1 mutations is a gain of a toxic function. The molecular mechanisms for this acquired adverse function are not known. If indeed the primary cause of the disease is oxidative cytotoxicity, the gained function presumable involves aberrant production or trafficking of one or more toxic oxidative intermediates.

Levels of free radicals are regulated by two major endogenous antioxidant systems: non-enzymatic free radical scavengers (vitamins E and C, beta-carotene and uric acid) and enzymes (SOD, catalase and glutathione peroxidase). Reactive oxygen species are highly reactive and typically short-lived. It is difficult to measure their levels directly. Accordingly, several biochemical parameters are used to gauge the extent of oxidative damage to various cellular constituents, including markers of oxidative damage to DNA, proteins and lipids. Protein oxidation can be quantitated by measuring protein carbonyl groups in plasma and in tissue. Protein carbonyl groups have been found to be increased in brains and spinal cords from sporadic ALS patients as compared to controls and patients with FALS.

### Role of Neuronal Over-Stimulation in Pathogenesis of Amyotrophic Lateral Sclerosis

Another theory regarding the etiology of ALS is that neuronal cell death in ALS is the result of over-excitement of neuronal cells due to excess extracellular glutamate. Glutamate is a neurotransmitter that is released by glutaminergic neurons and is taken up into glial cells where it is converted into glutamine by the enzyme glutamine synthetase. Glutamine then re-enters the neurons and is hydrolyzed by glutaminase to form glutamate, thus replenishing the neurotransmitter pool. In a normal spinal cord and brain stem, the level of extracellular glutamate is kept at low micromolar levels in the extracellular fluid because glial cells, which function in part to support neurons, use the excitatory amino acid transporter type 2 (EAAT2) protein to absorb glutamate immediately. A deficiency in the normal EAAT2 protein in patients with ALS was identified as being important in the pathology of the disease. One explanation for the reduced levels of EAAT2 is that EAAT2 is spliced aberrantly. The aberrant splicing produces a splice variant with a deletion of 45 to 107 amino acids located in the C-terminal region of the EAAT2 protein. Due to the lack of, or defectiveness of EAAT2, extracellular glutamate accumulates, causing neurons to fire continuously. The accumulation of glutamate has a toxic effect on neuronal cells because continual firing of the neurons leads to early cell death.

### Role of Proteasome or Protein Dysfunction in Pathogenesis of Amyotrophic Lateral Sclerosis

Additionally, evidence is accumulating that as a result of the normal aging process the body increasingly loses the ability to adequately degrade mutated or misfolded proteins. The proteasome is the piece of biological machinery responsible for most normal degradation of proteins inside cells. Age related loss of function or change of function of the proteasome may contribute to many neurodegenerative conditions, including ALS.

### Lack of Adequate Treatments for Amyotrophic Lateral Sclerosis

Presently, there is no cure for ALS, nor is there a therapy that has been proven effective to prevent or reverse the course of the disease. Attempts to treat ALS have involved treating neuronal degeneration with long-chain fatty alcohols which have cytoprotective effects, with a salt of pyruvic acid, or with glutamine synthetase to block the glutamate cascade. For example, Riluzole™, a glutamate release inhibitor, has been approved by the Food and Drug Administration in the U.S. for the treatment of ALS, and appears to extend the life of at least some patients with ALS. However, some reports have indicated that even though Riluzole™ therapy can prolong survival time, it does not appear to provide an improvement of muscular strength in the patients.

### Summary of Hydrogenated Pyrido[4,3-b]Indole Derivatives

Known compounds of the class of tetra- and hexahydro-1H-pyrido[4,3-b]indole derivatives manifest a broad spectrum of biological activity. In the series of 2,3,4,5-tetrahydro-1H-pyrido[4,3-b]indoles the following types of activity have been found: antihistamine activity (DE 1,813,229, filed Dec. 6, 1968; DE 1,952,800, filed Oct. 20, 1969), central depressive and anti-inflammatory activity (U.S. Pat. No. 3,718,657, filed Dec. 3, 1970), neuroleptic activity (Herbert C. A., Plattner S. S., Welch W. M.—Mol. Pharm. 1980, v. 17, N 1, p. 38-42) and others. 2,3,4,4a,5,9b-hexahydro-1H-pyrido[4,3-b]indole derivatives show psychotropic (Welch W. M., Harbert C. A., Weissman A., Koe B. K. J. Med. Chem., 1986, vol. 29, No. 10, p. 2093-2099), antiaggressive, antiarrhythmic and other types of activity.

Several drugs, such as diazoline (mebhydroline), dimebon, dorastine, carbidine (dicarbine), stobadine and gevotroline, based on tetra- or hexahydro-1H-pyrido[4,3-b]indole derivatives are known to have been manufactured. Diazoline (2-methyl-5-benzyl-2,3,4,5-tetrahydro-1H-pyrido[4,3-b]indole dihydrochloride) (Klyuev M. A., Drugs, used in “Medical Pract.”, USSR, Moscow, “Meditzina” Publishers, 1991, p. 512) and dimebon (2,8-dimethyl-5-(2-(6-methyl-3-pyridyl)ethyl-2,3,4,5-tetrahydro-1H-pyrido[4,3-b]indole dihydrochloride) (M. D. Mashkovsky, “Medicinal Drugs” in 2 vol. Vol. 1-12th Edition, Moscow, “Meditzina” Publishers, 1993, p. 383) as well as dorastine (2-methyl-8-chloro-5-[2-(6-methyl-3-pyridyl)ethyl]-2,3,4,5-tetrahydro-1H-pyrido[4,3-b]indole dihydrochloride) (USAN and USP dictionary of drugs names (United States Adopted Names, 1961-1988, current US Pharmacopoeia and National Formula for Drugs and other nonproprietary drug names), 1989, 26th Edition., p. 196) are known as antihistamine drugs; carbidine (dicarbine) (cis(±)-2,8-dimethyl-2,3,4,4a,5,9b-hexahydro-1H-pyrido[4,3-b]indole dihydrochloride) is a neuroleptic agent having an antidepressive effect (L. N. Yakhontov, R. G. Glushkov, Synthetic Drugs, ed. by A. G. Natradze, Moscow, “Meditzina” Publishers, 1983, p. 234-237), and its (−)isomer, stobadine, is known as an antiarrythmic agent (Kitlova M., Gibela P., Drimal J., Bratisl. Lek. Listy, 1985, vol. 84, No. 5, p. 542-549); gevotroline 8-fluoro-2-(3-(3-pyridyl)propyl)-2,3,4,5-tetrahydro-1H-pyrido[4,3-b]indole dihydrochloride is an antipsychotic and anxiolytic agent (Abou-Gharbi M., Patel U. R., Webb M. B., Moyer J. A., Ardnee T. H., J. Med. Chem., 1987, vol. 30, p. 1818-1823). Dimebon has been used in medicine as an antiallergic agent (Inventor's Certificate No. 1138164, IP Class A61K 31/47,5, C07 D 209/52, published on Feb. 7, 1985) in Russia for over 20 years.

As described in U.S. Pat. Nos. 6,187,785 and 7,021,206, hydrogenated pyrido[4,3-b]indole derivatives, such as dimebon, have NMDA antagonist properties, which make them useful for treating neurodegenerative diseases, such as Alzheimer's disease. As described in WO 2005/055951, hydrogenated pyrido[4,3-b]indole derivatives, such as dimebon, are useful as human or veterinary geroprotectors e.g., by delaying the onset and/or development of an age-associated or related manifestation and/or pathology or condition, including disturbance in skin-hair integument, vision disturbance and weight loss. U.S. patent application Ser. No. 11/543,529 (U.S. Publication No. 20070117835) and Ser. No. 11/543,341 (U.S. Publication No. 20070117834) disclose hydrogenated pyrido[4,3-b]indole derivatives, such as dimebon, as neuroprotectors for use in treating and/or preventing and/or slowing the progression or onset and/or development of Huntington's disease. WO 2007/087425, published Aug. 2, 2007, describes hydrogenated pyrido[4,3-b]indole derivatives, such as dimebon, for use in treating schizophrenia.

### Significant Medical Need

There remains a significant interest in and need for additional or alternative therapies for treating, preventing and/or delaying the onset and/or development of ALS. Preferably, the therapeutic agents can improve the quality of life and/or prolong the survival time for patients with ALS.

### BRIEF SUMMARY OF THE INVENTION

Methods, compounds and compositions for treating and/or preventing and/or delaying the onset and/or the development of ALS using a hydrogenated[4,3-b]indole or pharmaceutically acceptable salt thereof are described. The methods and compositions may comprise the compounds detailed herein, including without limitation the compound dimebon (2,8-dimethyl-5-(2-(6-methyl-3-pyridyl)ethyl)-2,3,4,5-tetrahydro-1H-pyrido[4,3-b]indole dihydrochloride).

In one embodiment, the present invention provides a method of treating ALS in an individual in need thereof by administering to the individual an effective amount of a hydrogenated pyrido(4,3-b)indole or pharmaceutically acceptable salt thereof. In another embodiment, the present invention provides a method of preventing or slowing the onset and/or development of ALS in an individual who has a mutated or abnormal gene associated with ALS (e.g., a SOD1 mutation). In another embodiment, the present invention provides a method of slowing the progression of ALS in an individual who has been diagnosed with ALS by administering to the individual an effective amount of a hydrogenated pyrido(4,3-b)indole or pharmaceutically acceptable salt thereof. In another embodiment, the present invention provides a method of preventing or slowing the onset and/or development of ALS in an individual who is at risk of developing ALS (e.g., an individual with a SOD1 mutation) by administering to the individual an effective amount of a hydrogenated pyrido(4,3-b)indole or pharmaceutically acceptable salt thereof. In any of the methods disclosed herein, the hydrogenated pyrido(4,3-b)indole may be dimebon.

In one aspect, the invention provides a unit dosage form comprising (a) first therapy comprising a hydrogenated pyrido(4,3-b)indole or pharmaceutically acceptable salt thereof, (b) a second therapy comprising another compound or pharmaceutically acceptable salt thereof that is useful for treating, preventing and/or delaying the onset and/or development of ALS and (c) a pharmaceutically acceptable carrier.

### BRIEF DESCRIPTION OF THE DRAWINGS

FIG. 1 illustrates the minimal toxicity of dimebon in Drosophila (fruit fly).

FIG. 2 illustrates dimebon's ability to suppress degeneration of photoreceptor neurons in a Drosophila (fruit fly) model.

FIG. 3 is a graph of the Kaplan-Meier estimates of time to reach stage 1 by treatment group for both sexes combined. Treatment started at day 85 after the onset of symptoms in this animal model.

FIG. 4 is a graph of the Kaplan-Meier estimates of time to reach stage 1 by treatment group for females.

FIG. 5 is a graph of the Kaplan-Meier estimates of time to reach stage 1 by treatment group for males.

FIG. 6 is a graph of the Kaplan-Meier estimates of time to reach stage 2 by treatment group for both sexes combined. Treatment started at day 85 after the onset of symptoms in this animal model.

FIG. 7 is a graph of the Kaplan-Meier estimates of time to reach stage 2 by treatment group for females.

FIG. 8 is a graph of the Kaplan-Meier estimates of time to reach stage 2 by treatment group for males.

FIG. 9 is a graph of the Kaplan-Meier estimates of time to reach stage 1 by treatment group for both sexes combined.

FIG. 10 is a graph of the Kaplan-Meier estimates of time to reach stage 2 by treatment group for both sexes combined.

FIG. 11 illustrates the effect of dimebon on ionomycin-induced toxicity of SK-N-SH cells.

FIG. 12 illustrates the effect of dimebon on ionomycin-induced toxicity of SY-SH5Y cells.

FIG. 13 illustrates the neuroprotective effects of dimebon on neuronal viability obtained in an in vitro 2% (growth factor withdrawal) assay. Neuronal viability was assessed at the end of the culture period with the MTT assay and results are shown as % of control (100%). Values represent the mean neuronal viability in percent and the sem from two independent experiments performed at two days with two 96-well plates (n=8).

### DETAILED DESCRIPTION OF THE INVENTION

### Definitions

For use herein, unless clearly indicated otherwise, use of the terms “a”, “an” and the like refers to one or more. It is also understood and clearly conveyed by this disclosure that reference to “the compound” or “a compound” includes and refers to any compound or pharmaceutically acceptable salt or other form thereof as described herein, such as the compound dimebon.

“Amyotrophic lateral sclerosis” or “ALS” are terms understood in the art and are used herein to denote a progressive neurodegenerative disease that affects upper motor neurons (motor neurons in the brain) and/or lower motor neurons (motor neurons in the spinal cord) and results in motor neuron death. As used herein, the term “ALS” includes all of the classifications of ALS known in the art, including, but not limited to classical ALS (typically affecting both lower and upper motor neurons), Primary Lateral Sclerosis (PLS, typically affecting only the upper motor neurons), Progressive Bulbar Palsy (PBP or Bulbar Onset, a version of ALS that typically begins with difficulties swallowing, chewing and speaking), Progressive Muscular Atrophy (PMA, typically affecting only the lower motor neurons) and familial ALS (a genetic version of ALS).

For use herein, unless clearly indicated otherwise, “an individual” as used herein intends a mammal, including but not limited to a human. The individual may be a human who has been diagnosed with or is suspected of having ALS. The individual may be a human who exhibits one or more symptoms associated with ALS. The individual may be a human who has a mutated or abnormal gene associated with ALS but who has not been diagnosed with ALS. The individual may be a human who is genetically or otherwise predisposed to developing ALS. In one variation, the individual is a human who has not been diagnosed with and/or is not considered at risk for developing Alzheimer's disease, Huntington's disease or schizophrenia. In one variation, the individual is a human who does not have a cognition impairment associated with aging or does not have a non-life threatening condition associated with the aging process (such as loss of sight (cataract), deterioration of the dermatohairy integument (alopecia) or an age-associated decrease in weight due to the death of muscular and fatty cells) or a combination thereof.

As used herein, an “at risk” individual is an individual who is at risk of development of ALS. An individual “at risk” may or may not have detectable disease, and may or may not have displayed detectable disease prior to the treatment methods described herein. “At risk” denotes that an individual has one or more so-called risk factors, which are measurable parameters that correlate with development of ALS. An individual having one or more of these risk factors has a higher probability of developing ALS than an individual without these risk factor(s). These risk factors include, but are not limited to, age, sex, race, diet, history of previous disease, presence of precursor disease, genetic (i.e., hereditary) considerations, and environmental exposure. Individuals at risk for ALS include, e.g., those having relatives who have experienced this disease, and those whose risk is determined by analysis of genetic or biochemical markers.

As used herein, “treatment” or “treating” is an approach for obtaining beneficial or desired results including clinical results. For purposes of this invention, beneficial or desired clinical results include, but are not limited to, one or more of the following: decreasing one more symptoms resulting from the disease, increasing the quality of life, decreasing the dose of one or more other medications required to treat the disease, delaying the progression of the disease, and/or prolonging survival. In some embodiments, an individual or combination therapy of the invention reduces the severity of one or more symptoms associated with ALS by at least 10, 20, 30, 40, 50, 60, 70, 80, 90, or 95% compared to the corresponding symptom in the same subject prior to treatment or compared to the corresponding symptom in other subjects not receiving the therapy.

As used herein, “delaying” development of ALS means to defer, hinder, slow, retard, stabilize and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of ALS is a method that reduces probability of disease development in a given time frame and/or reduces extent of the disease in a given time frame, when compared to not using the method. Such comparisons are typically based on clinical studies, using a statistically significant number of subjects. ALS development can be detectable using standard clinical techniques, such as standard neurological examination/imaging or patient interview. Development may also refer to disease progression that may be initially undetectable and includes occurrence, recurrence and onset.

As used herein, by “combination therapy” is meant a first therapy that includes one or more hydrogenated pyrido[4,3-b]indoles or pharmaceutically acceptable salts thereof in conjunction with a second therapy that includes one or more other compounds (or pharmaceutically acceptable salts thereof) or therapies (e.g., surgical procedures) useful for treating, preventing and/or delaying the onset and/or development of ALS. Administration in “conjunction with” another compound includes administration in the same or different composition, either sequentially, simultaneously, or continuously. In some embodiments, the combination therapy includes (i) one or more hydrogenated pyrido[4,3-b]indoles or pharmaceutically acceptable salts thereof and (ii) one or more agents that promote or increase the supply of energy to muscle cells, COX-2 inhibitors, poly(ADP-ribose)polymerase-1 (PARP-1) inhibitors, 30S ribosomal protein inhibitors, NMDA antagonists, NMDA receptor antagonists, sodium channel blockers, glutamate release inhibitors, K(V)4.3 channel blockers, anti-inflammatory agents, 5-HT1A receptor agonists, neurotrophic factor enhancers, agents that promote motoneuron phenotypic survival and/or neuritogenesis, agents that protect the blood brain barrier from disruption, inhibitors of the production or activity of one or more proinflammatory cytokines, immunomodulators, neuroprotectants, modulators of the function of astrocytes, antioxidants (such as small molecule catalytic antioxidants), free radical scavengers, agents that decrease the amount of one or more reactive oxygen species, agents that inhibit the decrease of non-protein thiol content, stimulators of a normal cellular protein repair pathway (such as agents that activate molecular chaperones), neurotrophic agents, inhibitors of nerve cell death, stimulators of neurite growth, agents that prevent the death of nerve cells and/or promote regeneration of damaged brain tissue, cytokine modulators, agents that reduce the level of activation of microglial cells, cannabinoid CB1 receptor ligands, non-steroidal anti-inflammatory drugs, cannabinoid CB2 receptor ligands, creatine, creatine derivatives, stereoisomers of a dopamine receptor agonist such as pramipexole hydrochloride, ciliary neurotrophic factors, agents that encode a ciliary neurotrophic factor, glial derived neurotrophic factors, agents that encode a glial derived neurotrophic factor, neurotrophin 3, agents that encode neurotrophin 3, or any combination of two or more of the foregoing

In some variations, the combination therapy optionally includes one or more pharmaceutically acceptable carriers or excipients, non-pharmaceutically active compounds, and/or inert substances.

As used herein, by “pharmaceutically active compound,” “pharmacologically active compound” or “active ingredient” is meant a chemical compound that induces a desired effect, e.g., treating and/or preventing and/or delaying the onset and/or the development of ALS.

The term “effective amount” intends such amount of a compound (e.g., a component of a combination therapy of the invention such as a compound described by the Formula (1), (2), (A), or (B) or a second therapy described herein) or a combination therapy, which in combination with its parameters of efficacy and toxicity, as well as based on the knowledge of the practicing specialist should be effective in a given therapeutic form. As is understood in the art, an effective amount may be in one or more doses, i.e., a single dose or multiple doses may be required to achieve the desired treatment endpoint. In some embodiments, the amount of the first therapy, the second therapy, or the combined therapy is an amount sufficient to modulate the amount or activity of one or more of the following: a muscle cell, COX-2, poly(ADP-ribose)polymerase-1 (PARP-1), 30S ribosomal protein, NMDA, NMDA receptor, sodium channel, glutamate, K(V)4.3 channel, inflammation, 5-HT1A receptor, neurotrophic factor, neuron, motoneuron phenotypic survival, neuritogenesis, disruption of the blood brain barrier, proinflammatory cytokine, immunomodulators, neuroprotectant, astrocyte, antioxidant, free radical scavenger, non-protein thiol content, normal cellular protein repair pathway, neurotrophic agent, nerve cell death, neurite growth, regeneration of damaged brain tissue, cytokine, microglial cell, cannabinoid CB1 receptor, cannabinoid CB1 receptor ligands, cannabinoid CB2 receptor, cannabinoid CB2 receptor ligands, creatine, creatine derivative, stereoisomer of a dopamine receptor agonist such as pramipexole hydrochloride, ciliary neurotrophic factor, glial derived neurotrophic factor, or neurotrophin 3. In some embodiments, one or more of these amounts or activities changes by at least or about 10%, 20%, 30%, 40%, 50%, 60%, 70%, 80%, 90%, 100%, 150%, 200%, 300%, 400%, 500% or more as compared to the corresponding amount or activity in the same subject prior to treatment or compared to the corresponding activity in other subjects not receiving the individual or combination therapy. Standard methods can be used to measure the magnitude of this effect, such as in vitro assays with purified enzyme, cell-based assays, animal models, or human testing.

As is understood in the clinical context, an effective dosage of a drug, compound or pharmaceutical composition that contains a compound described by the Formula (1) or by Formula (2) or any compound described herein (e.g., a compound described by the Formula (A) or (B)) may be achieved in conjunction with another drug, compound or pharmaceutical composition (such as a second therapy described herein). Thus, an effective amount may be considered in the context of administering one or more therapeutic agents, and a single agent may be considered to be given in an effective amount if, in conjunction with one or more other agents, a desirable or beneficial result may be or is achieved. The compounds in a combination therapy of the invention may be administered sequentially, simultaneously, or continuously using the same or different routes of administration for each compound. Thus, an effective amount of a combination therapy includes an amount of the first therapy and an amount of the second therapy that when administered sequentially, simultaneously, or continuously produces a desired outcome. Suitable doses of any of the coadministered compounds may optionally be lowered due to the combined action (e.g., additive or synergistic effects) of the compounds.

In various embodiments, treatment with the combination of the first and second therapies may result in an additive or even synergistic (e.g., greater than additive) result compared to administration of either therapy alone. In some embodiments, a lower amount of each pharmaceutically active compound is used as part of a combination therapy compared to the amount generally used for individual therapy. In some embodiments, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a pharmaceutically active compound in a combination therapy than the amount generally used for individual therapy. In some embodiments, the use of a small amount of pharmaceutically active compound results in a reduction in the number, severity, frequency, or duration of one or more side-effects associated with the compound.

A “therapeutically effective amount” refers to an amount of a compound or a combination therapy sufficient to produce a desired therapeutic outcome (e.g., reducing the severity or duration of, stabilizing the severity of, or eliminating one or more symptoms of ALS). For therapeutic use, beneficial or desired results include, e.g., clinical results such as decreasing one or more symptoms resulting from the disease (biochemical, histologic and/or behavioral), including its complications and intermediate pathological phenotypes presenting during development of the disease, increasing the quality of life of those suffering from the disease, decreasing the dose of other medications required to treat the disease, enhancing effect of another medication, delaying the progression of the disease and/or prolonging survival of patients.

A “prophylactically effective amount” refers to an amount of a compound or a combination therapy sufficient to prevent or reduce the severity of one or more future symptoms of ALS when administered to an individual who is susceptible and/or who may develop ALS. For prophylactic use, beneficial or desired results include, e.g., results such as eliminating or reducing the risk, lessening the severity, or delaying the onset of the disease, including biochemical, histologic and/or behavioral symptoms of the disease, its complications and intermediate pathological phenotypes presenting during development of the disease.

The term “simultaneous administration,” as used herein, means that a first therapy and second therapy in a combination therapy are administered with a time separation of no more than about 15 minutes, such as no more than about any of 10, 5, or 1 minutes. When the compounds are administered simultaneously, the first and second therapies may be contained in the same composition (e.g., a composition comprising both a hydrogenated pyrido[4,3-b]indole and a second therapy) or in separate compositions (e.g., a hydrogenated pyrido[4,3-b]indole is contained in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60 or more minutes. Either the first therapy or the second therapy may be administered first. The first and second therapies are contained in separate compositions, which may be contained in the same or different packages or kits.

The term “controlled release” refers to a drug-containing formulation or fraction thereof in which release of the drug is not immediate, i.e., with a “controlled release” formulation, administration does not result in immediate release of the drug into an absorption pool.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, by “activator,” “agonist,” or “enhancer” is meant an individual or combination therapy that increases the amount of or an activity of a biologically-active compound or cell, such as a muscle cell, 5-HT1A receptor, neurotrophic factor, motoneuron, molecular chaperone, non-protein thiol, cannabinoid CB1 receptor, cannabinoid CB2 receptor, creatine, creatine derivative, ciliary neurotrophic factor, glial derived neurotrophic factor, neurotrophin 3, or any combination of two or more of the foregoing. In some embodiments, the activator, agonist, or enhancer increases an activity by at least or about 10%, 20%, 30%, 40%, 50%, 60%, 70%, 80%, 90%, 100%, 150%, 200%, 300%, 400%, 500% or more as compared to the corresponding activity in the same subject prior to treatment or compared to the corresponding activity in other subjects not receiving the individual or combination therapy.

As used herein, by “inhibitor,” “antagonist,” or blocker is meant an individual or combination therapy that reduces or eliminates the amount of or an activity of a biologically-active compound or cell, such a COX-2 enzyme, poly(ADP-ribose)polymerase-1 (PARP-1), 30S ribosomal protein, NMDA, NMDA receptor, sodium channel, glutamate release, K(V)4.3 channel, inflammation, proinflammatory cytokine, free radical, reactive oxygen species, nerve cell death, microglial cells, or any combination of two or more of the foregoing. In some embodiments, the inhibitor, antagonist, or blocker reduces an activity by at least or about 10%, 20%, 30%, 40%, 50%, 60%, 70%, 80%, 90%, 95% or 100% as compared to the corresponding activity in the same subject prior to treatment or compared to the corresponding activity in other subjects not receiving the individual or combination therapy.

As used herein, by “modulator” is meant an individual or combination therapy that increases or decreases the amount of or an activity of a biologically-active compound or cell, such as a muscle cell, 5-HT1A receptor, neurotrophic factor, motoneuron, molecular chaperone, non-protein thiol, cannabinoid CB1 receptor, cannabinoid CB2 receptor, creatine, creatine derivative, ciliary neurotrophic factor, glial derived neurotrophic factor, neurotrophin 3, COX-2 enzyme, poly(ADP-ribose)polymerase-1 (PARP-1), 30S ribosomal protein, NMDA, NMDA receptor, sodium channel, glutamate release, K(V)4.3 channel, inflammation, proinflammatory cytokine, free radical, reactive oxygen species, nerve cell death, microglial cells, cytokine, astrocytes, or any combination of two or more of the foregoing. In some embodiments, the compound alters an activity by at least or about 10%, 20%, 30%, 40%, 50%, 60%, 70%, 80%, 90%, 100%, 150%, 200%, 300%, 400%, 500% or more as compared to the corresponding activity in the same subject prior to treatment or compared to the corresponding activity in other subjects not receiving the individual or combination therapy.

As used herein, a “NMDA receptor antagonist” is an individual or combination therapy that reduces or eliminates an activity of an N-methyl-D-aspartate (NMDA) receptor, which is an ionotropic receptor for glutamate. NMDA receptors bind both glutamate and the co-agonist glycine. Thus, an NMDA receptor antagonist can inhibit the ability of glutamate and/or glycine to activate an NMDA receptor. In some embodiments, the NMDA receptor antagonist binds to the active site of an NDMA receptor (e.g., a binding site for glutamate and/or glycine) or binds to an allosteric site on the receptor. The interaction between the NMDA receptor antagonist and the NMDA receptor may be reversible or irreversible. In some embodiments, the antagonist reduces an activity of an NMDA receptor by at least or about 10%, 20%, 30%, 40%, 50%, 60%, 70%, 80%, 90%, 95% or 100% as compared to the corresponding activity in the same subject prior to treatment or compared to the corresponding activity in other subjects not receiving the individual or combination therapy. Exemplary NMDA receptor antagonists include Memantine (Namenda® sold by Forest, Axura® sold by Merz, Akatinol® sold by Merz, Ebixa® sold by Lundbeck), Neramexane (Forest Labs), Amantadine, AP5 (2-amino-5-phosphonopentanoate, APV), Dextrorphan, Ketamine, MK-801 (dizocilpine), Phencyclidine, Riluzole and 7-chlorokynurenate. The structure of Neramexane is distinct from that of Namenda but they are pharmacologically equivalent.

As used herein, by “anti-inflammatory agent’ is meant an individual or combination therapy that reduces or eliminates inflammation. In some embodiments, the compound reduces inflammation by at least or about 10%, 20%, 30%, 40%, 50%, 60%, 70%, 80%, 90%, 95% or 100%.

### Methods for Treating Amyotrophic Lateral Sclerosis

The hydrogenated pyrido[4,3-b]indoles described herein may be used to treat, prevent and/or delay the onset and/or the development of ALS in mammals, such as humans. As illustrated in Example 1, the representative hydrogenated pyrido[4,3-b]indole dimebon did not show significant toxicity in a Drosophila model for toxicity at doses below 1 mM. Additionally, dimebon showed a neuroprotective effect in a Drosophila model of Huntington's disease (Example 2). This result supports the ability of the hydrogenated pyrido[4,3-b]indoles described herein to inhibit neuronal cell death, which is a characteristic of ALS. Exemplary methods for determining the ability of hydrogenated pyrido[4,3-b]indoles to treat or prevent ALS are described in Examples 3-4 and further methods are detailed in the experimental section.

Thus, the present invention provides a variety of methods, such as those described in the “Brief Summary of the Invention” and elsewhere in this disclosure. The methods of the invention employ the compounds described herein. For example, in one embodiment, the present invention provides a method of treating ALS in a patient in need thereof comprising administering to the individual an effective amount of a hydrogenated pyrido(4,3-b)indole, such as dimebon or pharmaceutically acceptable salt thereof. In one embodiment, the present invention provides a method of delaying the onset and/or development of ALS in an individual who is considered at risk for developing ALS (e.g., an individual whose one or more family members have had ALS or an individual who has been diagnosed as having a genetic mutation associated with ALS) comprising administering to the individual an effective amount of a hydrogenated pyrido(4,3-b)indole, such as dimebon or pharmaceutically acceptable salt thereof. In one embodiment, the present invention provides a method of delaying the onset and/or development of ALS in an individual who is genetically predisposed to developing ALS comprising administering to the individual an effective amount of a hydrogenated pyrido(4,3-b)indole, such as dimebon or pharmaceutically acceptable salt thereof. In one embodiment, the present invention provides a method of delaying the onset and/or development of ALS in an individual having a mutated or abnormal gene associated with ALS (e.g., a SOD1 mutation) but who has not been diagnosed with ALS comprising administering to the individual an effective amount of a hydrogenated pyrido(4,3-b)indole, such as dimebon or pharmaceutically acceptable salt thereof. In one embodiment, the present invention provides a method of preventing ALS in an individual who is genetically predisposed to developing ALS or who has a mutated or abnormal gene associated with ALS but who has not been diagnosed with ALS comprising administering to the individual an effective amount of a hydrogenated pyrido(4,3-b)indole, such as dimebon or pharmaceutically acceptable salt thereof. In one embodiment, the present invention provides a method of preventing the onset and/or development of ALS in an individual who is not identified as genetically predisposed to developing ALS comprising administering to the individual an effective amount of a hydrogenated pyrido(4,3-b)indole, such as dimebon or pharmaceutically acceptable salt thereof. In one embodiment, the present invention provides a method of decreasing the intensity or severity of the symptoms of ALS in an individual who is diagnosed with ALS comprising administering to the individual an effective amount of a hydrogenated pyrido(4,3-b)indole, such as dimebon or pharmaceutically acceptable salt thereof. In one embodiment, the present invention provides a method of increasing the survival time of an individual diagnosed with ALS comprising administering to the individual an effective amount of a hydrogenated pyrido(4,3-b)indole, such as dimebon or pharmaceutically acceptable salt thereof. In one embodiment, the present invention provides a method of enhancing the quality of life of an individual diagnosed with ALS comprising administering to the individual an effective amount of a hydrogenated pyrido(4,3-b)indole, such as dimebon or pharmaceutically acceptable salt thereof. In one variation, the method comprises the manufacture of a medicament for use in any of the above methods, e.g., treating and/or preventing and/or delaying the onset or development of ALS in a human.

### Compounds for Use in the Methods, Formulations, Kits and Inventions Discloses Herein

When reference to organic residues or moieties having a specific number of carbons is made, unless clearly stated otherwise, it intends all geometric isomers thereof. For example, “butyl” includes n-butyl, sec-butyl, isobutyl and t-butyl; “propyl” includes n-propyl and isopropyl.

The term “alkyl” intends and includes linear, branched or cyclic hydrocarbon structures and combinations thereof. Preferred alkyl groups are those having 20 carbon atoms (C20) or fewer. More preferred alkyl groups are those having fewer than 15 or fewer than 10 or fewer than 8 carbon atoms.

The term “lower alkyl” refers to alkyl groups of from 1 to 5 carbon atoms. Examples of lower alkyl groups include methyl, ethyl, propyl, isopropyl, butyl, s- and t-butyl and the like. Lower alkyl is a subset of alkyl.

The term “aryl” refers to an unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxain-3(4H)-one-7-yl), and the like. Preferred aryls includes phenyl and naphthyl.

The term “heteroaryl” refers to an aromatic carbocyclic group of from 2 to 10 carbon atoms and 1 to 4 heteroatoms selected from oxygen, nitrogen and sulfur within the ring. Such heteroaryl groups can have a single ring (e.g., pyridyl or furyl) or multiple condensed rings (e.g., indolizinyl or benzothienyl). Examples of heteroaryl residues include, e.g., imidazolyl, pyridinyl, indolyl, thiopheneyl, thiazolyl, furanyl, benzimidazolyl, quinolinyl, isoquinolinyl, pyrimidinyl, pyrazinyl, tetrazolyl and pyrazolyl.

The term “aralkyl” refers to a residue in which an aryl moiety is attached to the parent structure via an alkyl residue. Examples are benzyl, phenethyl and the like.

The term “heteroaralkyl” refers to a residue in which a heteroaryl moiety is attached to the parent structure via an alkyl residue. Examples include furanylmethyl, pyridinylmethyl, pyrimidinylethyl and the like.

The term “substituted heteroaralkyl” refers to heteroaryl groups which are substituted with from 1 to 3 substituents, such as residues selected from the group consisting of hydroxy, alkyl, alkoxy, alkenyl, alkynyl, amino, aryl, carboxyl, halo, nitro and amino.

The term “halo” or “halogen” refers to fluoro, chloro, bromo and iodo.

Compounds for use in the systems, methods and kits described herein are hydrogenated pyrido[4,3-b]indoles or pharmaceutically acceptable salts thereof, such as an acid or base salt thereof. A hydrogenated pyrido[4,3-b]indole can be a tetrahydro pyrido[4,3-b]indole or pharmaceutically acceptable salt thereof. The hydrogenated pyrido[4,3-b]indole can also be a hexahydro pyrido[4,3-b]indole or pharmaceutically acceptable salt thereof. The hydrogenated pyrido[4,3-b]indole compounds can be substituted with 1 to 3 substituents, although unsubstituted hydrogenated pyrido[4,3-b]indole compounds or hydrogenated pyrido[4,3-b]indole compounds with more than 3 substituents are also contemplated. Suitable substituents include but are not limited to alkyl, lower alkyl, aralkyl, heteroaralkyl, substituted heteroaralkyl, and halo.

Particular hydrogenated pyrido-([4,3-b])indoles are exemplified by the Formulae A and B:

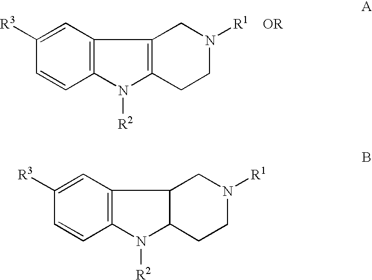

where R¹ is selected from the group consisting of alkyl, lower alkyl and aralkyl, R² is selected from the group consisting of hydrogen, aralkyl and substituted heteroaralkyl; and R³ is selected from the group consisting of hydrogen, alkyl, lower alkyl and halo.

In one variation, R¹ is alkyl, such as an alkyl selected from the group consisting of C₁-C₁₅alkyl, C₁₀-C₁₅alkyl, C₁-C₁₀alkyl, C₂-C₁₅alkyl, C₂-C₁₀alkyl, C₂-C₈alkyl, C₄-C₈alkyl, C₆-C₈alkyl, C₆-C₁₅alkyl, C₁₅-C₂₀alkyl; C₁-C₈alkyl and C₁-C₆alkyl. In one variation, R¹ is aralkyl. In one variation, R¹ is lower alkyl, such as a lower alkyl selected from the group consisting of C₁-C₂alkyl, C₁-C₄alkyl, C₂-C₄ alkyl, C₁-C₅ alkyl, C₁-C₃alkyl, and C₂-C₅alkyl.

In one variation, R¹ is a straight chain alkyl group. In one variation, R¹ is a branched alkyl group. In one variation, R¹ is a cyclic alkyl group.

In one variation, R¹ is methyl. In one variation, R¹ is ethyl. In one variation, R¹ is methyl or ethyl. In one variation, R¹ is methyl or an aralkyl group such as benzyl. In one variation, R¹ is ethyl or an aralkyl group such as benzyl.

In one variation, R¹ is an aralkyl group. In one variation, R¹ is an aralkyl group where any one of the alkyl or lower alkyl substituents listed in the preceding paragraphs is further substituted with an aryl group (e.g., Ar—C₁-C₆alkyl, Ar—C₁-C₃alkyl or Ar—C₁-C₁₅alkyl). In one variation, R¹ is an aralkyl group where any one of the alkyl or lower alkyl substituents listed in the preceding paragraphs is substituted with a single ring aryl residue. In one variation, R¹ is an aralkyl group where any one of the alkyl or lower alkyl substituents listed in the preceding paragraphs is further substituted with a phenyl group (e.g., Ph-C₁-C₆Alkyl or Ph-C₁-C₃Alkyl, Ph-C₁-C₁₅alkyl). In one variation, R¹ is benzyl.

All of the variations for R¹ are intended and hereby clearly described to be combined with any of the variations stated below for R² and R³ the same as if each and every combination of R¹, R² and R³ were specifically and individually listed.

In one variation, R² is H. In one variation, R² is an aralkyl group. In one variation, R² is a substituted heteroaralkyl group. In one variation, R² is hydrogen or an aralkyl group. In one variation, R² is hydrogen or a substituted heteroaralkyl group. In one variation, R² is an aralkyl group or a substituted heteroaralkyl group. In one variation, R² is selected from the group consisting of hydrogen, an aralkyl group and a substituted heteroaralkyl group.

In one variation, R² is an aralkyl group where R² can be any one of the aralkyl groups noted for R¹ above, the same as if each and every aralkyl variation listed for R¹ is separately and individually listed for R².

In one variation, R² is a substituted heteroaralkyl group, where the alkyl moiety of the heteroaralkyl can be any alkyl or lower alkyl group, such as those listed above for R¹. In one variation, R² is a substituted heteroaralkyl where the heteroaryl group is substituted with 1 to 3 C₁-C₃ alkyl substituents (e.g., 6-methyl-3-pyridylethyl). In one variation, R² is a substituted heteroaralkyl group wherein the heteroaryl group is substituted with 1 to 3 methyl groups. In one variation, R² is a substituted heteroaralkyl group wherein the heteroaryl group is substituted with one lower alkyl substituent. In one variation, R² is a substituted heteroaralkyl group wherein the heteroaryl group is substituted with one C₁-C₃ alkyl substituent. In one variation, R² is a substituted heteroaralkyl group wherein the heteroaryl group is substituted with one or two methyl groups. In one variation, R² is a substituted heteroaralkyl group wherein the heteroaryl group is substituted with one methyl group.

In other variations, R² is any one of the substituted heteroaralkyl groups in the immediately preceding paragraph where the heteroaryl moiety of the heteroaralkyl group is a single ring heteroaryl group. In other variations, R² is any one of the substituted heteroaralkyl groups in the immediately preceding paragraph where the heteroaryl moiety of the heteroaralkyl group is a multiple condensed ring heteroaryl group. In other variations, R² is any one of the substituted heteroaralkyl groups in the immediately preceding paragraph where the heteroaralkyl moiety is a pyridyl group (Py).

In one variation, R² is 6-CH₃-3-Py-(CH₂)₂—.

In one variation, R³ is hydrogen. In other variations, R³ is any one of the alkyl groups noted for R¹ above, the same as if each and every alkyl variation listed for R¹ is separately and individually listed for R³. In another variation, R³ is a halo group. In one variation, R³ is hydrogen or an alkyl group. In one variation, R³ is a halo or alkyl group. In one variation, R³ is hydrogen or a halo group. In one variation, R³ is selected from the group consisting of hydrogen, alkyl and halo. In one variation, R³ is Br. In one variation, R³ is I. In one variation, R³ is F. In one variation, R³ is Cl.

In a particular variation, the hydrogenated pyrido[4,3-b]indole is 2,8-dimethyl-5-(2-(6-methyl-3-pyridyl)ethyl)-2,3,4,5-tetrahydro-1H-pyrido[4,3-b]indole or a pharmaceutically acceptable salt thereof.

The hydrogenated pyrido[4,3-b]indoles can be in the form of pharmaceutically acceptable salts thereof, which are readily known to those of skill in the art. The pharmaceutically acceptable salts include pharmaceutically acceptable acid salts. Examples of particular pharmaceutically acceptable salts include hydrochloride salts or dihydrochloride salts. In a particular variation, the hydrogenated pyrido[4,3-b]indole is a pharmaceutically acceptable salt of 2,8-dimethyl-5-(2-(6-methyl-3-pyridyl)ethyl)-2,3,4,5-tetrahydro-1H-pyrido[4,3-b]indole, such as 2,8-dimethyl-5-(2-(6-methyl-3-pyridyl)ethyl)-2,3,4,5-tetrahydro-1H-pyrido[4,3-b]indole dihydrochloride (dimebon).

Particular hydrogenated pyrido-([4,3-b])indoles can also be described by the Formula (1) or by the Formula (2):

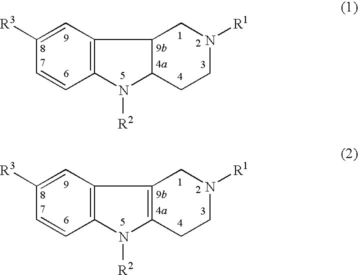

For compounds of a general Formula (1) or (2),

R¹ represents —CH₃, CH₃CH₂—, or PhCH₂-(benzyl);

R² is —H, PhCH₂—, or 6CH₃-3-Py-(CH2)₂-;

R³ is —H, —CH₃, or —Br,

in any combination of the above substituents. All possible combinations of the substituents of Formula (1) and (2) are contemplated as specific and individual compounds the same as if each single and individual compound were listed by chemical name. Also contemplated are the compounds of Formula (1) or (2), with any deletion of one or more possible moieties from the substituent groups listed above: e.g., where R¹ represents —CH₃; R² is —H, PhCH₂—, or 6CH₃-3-Py-(CH₂)₂—; and R³ is —H, —CH₃, or —Br, or where R¹ represents —CH₃; R² is 6CH₃-3-Py-(CH₂)₂—; and R³ represents —H, —CH₃, or —Br.

The above and any compound herein may be in a form of salts with pharmaceutically acceptable acids and in a form of quaternized derivatives.

The compound may be Formula (1), where R¹ is —CH₃, R² is —H, and R³ is —CH₃. The compound may be Formula (2), where R¹ is represented by —CH₃, CH₃CH₂—, or PhCH₂—; R² is —H, PhCH₂—, or 6CH₃-3-Py-(CH₂)₂—; R³ is —H, —CH₃, or —Br. The compound may be Formula (2), where R¹ is CH₃CH₂— or PhCH₂—, R² is —H, and R³ is —H; or a compound, where R¹ is —CH₃, R² is PhCH₂—, R³ is —CH₃; or a compound, where R¹ is —CH₃, R² is 6-CH₃-3-Py-(CH₂)₂—, and R³ is —CH₃; or a compound, where R¹ is —CH₃, R² is —H, R³ is —H or —CH₃; or a compound, where R¹ is —CH₃, R² is —H, R³ is —Br.

Compounds known from literature which can be used in the methods disclosed herein include the following specific compounds:

1. cis(±) 2,8-dimethyl-2,3,4,4a,5,9b-hexahydro-1H-pyrido[4,3-b]indole and its dihydrochloride;

2. 2-ethyl-2,3,4,5-tetrahydro-1H-pyrido[4,3-b]indole;

3. 2-benzyl-2,3,4,5-tetrahydro-1H-pyrido[4,3-b]indole;

4. 2,8-dimethyl-5-benzyl-2,3,4,5-tetrahydro-1H-pyrido[4,3-b]indole and its dihydrochloride;

5. 2-methyl-5-(2-methyl-3-pyridyl)ethyl-2,3,4,5-tetrahydro-1H-pyrido[4,3-b]indole and its sesquisulfate;

6. 2, 8-dimethyl-5-(2-(6-methyl-3-pyridyl)ethyl)-2,3,4,5-tetrahydro-1H-pyrido[4,3-b]indole and its dihydrochloride (dimebon);

7. 2-methyl-2,3,4,5-tetrahydro-1H-pyrido[4,3-b]indole;

8. 2,8-dimethyl-2,3,4,5-tetrahydro-1H-pyrido[4,3-b]indole and its methyl iodide;

9. 2-methyl-8-bromo-2,3,4,5-tetrahydro-1H-pyrido[4,3-b]indole and its hydrochloride.

In one variation, the compound is of the Formula A or B and R¹ is selected from a lower alkyl or benzyl; R² is selected from a hydrogen, benzyl or 6-CH₃-3-Py-(CH₂)₂— and R³ is selected from hydrogen, lower alkyl or halo, or any pharmaceutically acceptable salt thereof. In another variation, R¹ is selected from —CH₃, CH₃CH₂—, or benzyl; R² is selected from —H, benzyl, or 6-CH₃-3-Py-(CH₂)₂—; and R³ is selected from —H, —CH₃ or —Br, or any pharmaceutically acceptable salt thereof. In another variation the compound is selected from the group consisting of: cis(±) 2,8-dimethyl-2,3,4,4a,5,9b-hexahydro-1H-pyrido[4,3-b]indole as a racemic mixture or in the substantially pure (+) or substantially pure (−) form; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole; 2-methyl-5-(2-methyl-3-pyridyl)ethyl-2,3,4,5-tetrahydro-1H-pyrido[4,3-b]indole; 2,8-dimethyl-5-(2-(6-methyl-3-pyridyl)ethyl)-2,3,4,5-tetrahydro-1H-pyrido[4,3-b]indole; 2-methyl-2,3,4,5-tetrahydro-1H-pyrido[4,3-b]indole; 2,8-dimethyl-2,3,4,5-tetrahydro-1H-pyrido[4,3-b]indole; or 2-methyl-8-bromo-2,3,4,5-tetrahydro-1H-pyrido[4,3-b]indole or any pharmaceutically acceptable salt of any of the foregoing. In one variation, the compound is of the formula A or B wherein R¹ is —CH₃, R² is —H and R³ is —CH₃ or any pharmaceutically acceptable salt thereof. The compound may be of the Formula A or B where R¹ CH₃CH₂— or benzyl, R² is —H, and R³ is —CH₃ or any pharmaceutically acceptable salt thereof. The compound may be of the Formula A or B where R¹ is —CH₃, R² is benzyl, and R³ is —CH₃ or any pharmaceutically acceptable salt thereof. The compound may be of the Formula A or B where R¹ is —CH₃, R² is 6-CH₃-3-Py-(CH₂)₂—, and R³ is —H or any pharmaceutically acceptable salt thereof. The compound may be of the Formula A or B where R² is 6-CH₃-3-Py-(CH₂)₂— or any pharmaceutically acceptable salt thereof. The compound may be of the Formula A or B where R¹ is —CH₃, R² is —H, and R³ is —H or —CH₃ or any pharmaceutically acceptable salt, thereof. The compound may be of the Formula A or B where R¹ is —CH₃, R² is —H, and R³ is —Br, or any pharmaceutically acceptable salt thereof. The compound may be of the Formula A or B where R¹ is selected from a lower alkyl or aralkyl, R² is selected from a hydrogen, aralkyl or substituted heteroaralkyl and R³ is selected from hydrogen, lower alkyl or halo.

The compound for use in the systems and methods may be 2,8-dimethyl-5-(2-(6-methyl-3-pyridyl)ethyl-2,3,4,5-tetrahydro-1H-pyrido[4,3-b]indole or any pharmaceutically acceptable salt thereof, such as an acid salt, a hydrochloride salt or a dihydrochloride salt thereof.

Any of the compounds disclosed herein having two stereocenters in the pyrido[4,3-b]indole ring structure (e.g., carbons 4a and 9b of compound (1)) includes compounds whose stereocenters are in a cis or a trans form. A composition may comprise such a compound in substantially pure form, such as a composition of substantially pure S,S or R,R or S,R or R,S compound. A composition of substantially pure compound means that the composition contains no more than 15% or no more than 10% or no more than 5% or no more than 3% or no more than 1% impurity of the compound in a different stereochemical form. For instance, a composition of substantially pure S,S compound means that the composition contains no more than 15% or no more than 10% or no more than 5% or no more than 3% or no more than 1% of the R,R or S,R or R,S form of the compound. A composition may contain the compound as mixtures of such stereoisomers, where the mixture may be enanteomers (e.g., S,S and R,R) or diastereomers (e.g., S,S and R,S or S,R) in equal or unequal amounts. A composition may contain the compound as a mixture of 2 or 3 or 4 such stereoisomers in any ratio of stereoisomers. Compounds disclosed herein having stereocenters other than in the pyrido[4,3-b]indole ring structure intends all stereochemical variations of such compounds, including but not limited to enantiomers and diastereomers in any ratio, and includes racemic and enantioenriched and other possible mixtures. Unless stereochemistry is explicitly indicated in a structure, the structure is intended to embrace all possible stereoisomers of the compound depicted.

Synthesis and studies on neuroleptic properties for cis(±) 2,8-dimethyl-2,3,4,4a,5,9b-hexahydro-1H-pyrido[4,3-b]indole and its dihydrochloride are reported, for instance, in the following publication: Yakhontov, L. N., Glushkov, R. G., Synthetic therapeutic drugs. A. G. Natradze, the editor, Moscow Medicina, 1983, p. 234-237. Synthesis of compounds 2, 8, and 9 above, and data on their properties as serotonin antagonists are reported in, for instance, in C. J. Cattanach, A. Cohen & B. H. Brown in J. Chem. Soc. (Ser.C) 1968, p. 1235-1243. Synthesis of the compound 3 above is reported, for instance, in the article N. P. Buu-Hoi, O. Roussel, P. Jacquignon, J. Chem. Soc., 1964, N 2, p. 708-711. N. F. Kucherova and N. K. Kochetkov (General chemistry (russ.), 1956, v. 26, p. 3149-3154) describe the synthesis of the compound 4 above. Synthesis of compounds 5 and 6 above is described in the article by A. N. Kost, M. A. Yurovskaya, T. V. Mel'nikova, in Chemistry of heterocyclic compounds, 1973, N 2, p. 207-212. The synthesis of the compound 7 above is described by U, Horlein in Chem. Ber., 1954, Bd. 87, hft 4, 463-p. 472. M. Yurovskaya and I. L. Rodionov in Chemistry of heterocyclic compounds (1981, N 8, p. 1072-1078) describe the synthesis of methyl iodide of the compound 8 above.

### Exemplary Combination Therapies

The invention also features combination therapies that include a first therapy comprising a hydrogenated pyrido[4,3-b]indole (such as a compound described by the Formula (1), (2), (A) or (B)) and a second therapy comprising one or more other compounds (such as a compound or pharmaceutically acceptable salt thereof that is useful for treating, preventing and/or delaying the onset and/or development of ALS).

Exemplary second therapies comprise one or more of the following compounds: agents that promote or increase the supply of energy to muscle cells, COX-2 inhibitors, poly(ADP-ribose)polymerase-1 (PARP-1) inhibitors, 30S ribosomal protein inhibitors, NMDA antagonists, NMDA receptor antagonists, sodium channel blockers, glutamate release inhibitors, K(V)4.3 channel blockers, anti-inflammatory agents, 5-HT1A receptor agonists, neurotrophic factor enhancers, agents that promote motoneuron phenotypic survival and/or neuritogenesis, agents that protect the blood brain barrier from disruption, inhibitors of the production or activity of one or more proinflammatory cytokines, immunomodulators, neuroprotectants, modulators of the function of astrocytes, antioxidants (such as small molecule catalytic antioxidants), free radical scavengers, agents that decrease the amount of one or more reactive oxygen species, agents that inhibit the decrease of non-protein thiol content, stimulators of a normal cellular protein repair pathway (such as agents that activate molecular chaperones), neurotrophic agents, inhibitors of nerve cell death, stimulators of neurite growth, agents that prevent the death of nerve cells and/or promote regeneration of damaged brain tissue, cytokine modulators, agents that reduce the level of activation of microglial cells, cannabinoid CB1 receptor ligands, non-steroidal anti-inflammatory drugs, cannabinoid CB2 receptor ligands, creatine, creatine derivatives, stereoisomers of a dopamine receptor agonist such as pramipexole hydrochloride, ciliary neurotrophic factors, agents that encode a ciliary neurotrophic factor, glial derived neurotrophic factors, agents that encode a glial derived neurotrophic factor, neurotrophin 3, agents that encode neurotrophin 3, and any combination of two or more of the foregoing.

In some embodiments, the second therapy includes two or more compounds that each has an activity that the other compound(s) does not have. In some embodiments, the second therapy includes one compound that has two or more different activities, such as a compound that functions as two or more of the following: an agent that promotes or increases the supply of energy to muscle cells, a COX-2 inhibitor, a poly(ADP-ribose)polymerase-1 (PARP-1) inhibitor, a 30S ribosomal protein inhibitor, an NMDA antagonist, an NMDA receptor antagonist, a sodium channel blocker, a glutamate release inhibitor, a K(V)4.3 channel blocker, anti-inflammatory agent, a 5-HT1A receptor agonist, a neurotrophic factor enhancer, an agent that promotes motoneuron phenotypic survival and/or neuritogenesis, an agent that protects the blood brain barrier from disruption, an inhibitor of the production or activity of one or more proinflammatory cytokines, an immunomodulator, a neuroprotectant, a modulator of the function of astrocytes, an antioxidant (such as a small molecule catalytic antioxidant), a free radical scavenger, an agent that decreases the amount of one or more reactive oxygen species, an agent that inhibits the decrease of non-protein thiol content, a stimulator of a normal cellular protein repair pathway (such as an agent that activates molecular chaperones), a neurotrophic agent, an inhibitor of nerve cell death, a stimulator of neurite growth, an agent that prevents the death of nerve cells and/or promotes regeneration of damaged brain tissue, a cytokine modulator, an agent that reduces the level of activation of microglial cells, a cannabinoid CB1 receptor ligand, a non-steroidal anti-inflammatory drug, a cannabinoid CB2 receptor ligand, creatine, a creatine derivative, a stereoisomer of a dopamine receptor agonist such as pramipexole hydrochloride, a ciliary neurotrophic factor, an agent that encodes a ciliary neurotrophic factor, a glial derived neurotrophic factor, an agent that encodes a glial derived neurotrophic factor, neurotrophin 3, and an agent that encodes neurotrophin 3.

An exemplary creatine that promotes or increases the supply of energy to muscle cells is ALS-02. ALS-02 is a therapeutic that incorporates an ultra-pure, clinical form of creatine. Avicena's lead drug candidate, ALS-02 is currently in phase III clinical trials for the treatment of ALS. Creatine is a nitrogenous organic acid that naturally occurs in vertebrates and helps to supply energy to muscle cells. ALS-02 was granted orphan drug designation by the FDA in February 2002 for the treatment of ALS.

An exemplary creatine derivative is ALS-08. ALS-08 is creatine derivative produced by Avicena that is in phase II clinical trials for the treatment of ALS in combination with the COX-2 inhibitor celecoxib or minocycline. ALS-08/celecoxib and ALS-08/minocycline combinations have demonstrated additive effects in animal models of ALS, reducing neurodegeneration and prolonging survival more than individual agents alone.

An exemplary poly(ADP-ribose)polymerase-1 (PARP-1) inhibitor and 30S ribosomal protein inhibitor is minocycline. Minocycline is thought to act by inhibiting microglial activation, inhibiting caspase activation, and thereby inhibiting apoptosis.

An exemplary and non-limiting list of NMDA receptor antagonists includes Memantine (Namenda® sold by Forest, Axura® sold by Merz, Akatinol® sold by Merz, Ebixa® sold by Lundbeck), Neramexane (Forest Labs), Amantadine, AP5 (2-amino-5-phosphonopentanoate, APV), Dextrorphan, Ketamine, MK-801 (dizocilpine), Phencyclidine, Riluzole and 7-chlorokynurenate. The structure of Neramexane is distinct from that of Namenda but they are pharmacologically equivalent.

An exemplary sodium channel blocker, glutamate release inhibitor, and K(V)4.3 channel blocker is Riluzole. Riluzole is thought to act on multiple pathways that minimize glutamate excitotoxicity and neuronal toxicity.

An exemplary anti-inflammatory agent is Procysteine. Anti-inflammatory agents may decrease microglial activation, cytokine release, inflammatory mediators, and/or cellular injury.

An exemplary 5-HT1A receptor agonist, neurotrophic factor enhancer, agent that promotes motoneuron phenotypic survival and/or neuritogenesis, and agent that protects the blood brain barrier from disruption is Xaliproden. This compound is reported to promote motoneuron phenotypic survival and neuritogenesis while protecting the blood brain barrier from disruption, which may be a result of the inhibition of production of proinflammatory cytokines. In January 2001, Xaliproden received orphan drug designation in the E.U. for the treatment of ALS.

An exemplary ciliary neurotrophic factor is recombinant human ciliary neurotrophic factor. Ciliary neurotrophic factors may improve neurite outgrowth, maintain neuronal structural integrity, regulate neuronal differentiation, and/or improve neuronal survival.

An exemplary immunomodulator therapy is Glatiramer acetate, such as Copolymer-1, Glatiramer acetate, also referred to as Copaxone®). Teva is conducting phase II trials for the treatment of ALS using this compound. The company is evaluating an oral formulation preclinically.

An exemplary neuroprotectant and modulator of the function of astrocytes is Arundic acid. Arundic acid is in phase II trials at Ono for the oral treatment of ALS. Arundic acid is believed to modulate the function of astrocytes.

An exemplary antioxidant and free radical scavenger is AEOL-10150, MnDTEIP. AEOL-10150 is a small molecule catalytic antioxidant in phase I trials at Aeolus Pharmaceuticals for the intravenous treatment of ALS. This compound scavenges a broad range reactive oxygen species that initiate an inflammatory cascade believed to be responsible for the degeneration of both upper and lower motor neurons in ALS. The compound has shown effectiveness in treating the symptoms of ALS in preclinical animal models.

An exemplary stimulator of a normal cellular protein repair pathway is Arimoclomol maleate. Arimoclomol maleate is currently undergoing phase II clinical trials at CytRx for the oral treatment of ALS. The compound is believed to function by a mechanism that stimulates a normal cellular protein repair pathway through the activation of molecular chaperones.

An exemplary neurotrophic agent, inhibitor of nerve cell death, stimulator of neurite growth, agent that decreases the amount of one or more reactive oxygen species, and agent that inhibits the decrease of non-protein thiol content is T-817 (1-[3-[2-(1-Benzothien-5-yl)ethoxy]propyl]azetidin-3-ol maleate). This compound inhibits nerve cell death and stimulates neurite growth. In preclinical trials, T-817MA also reduced oxidative stress by retarding an early sodium nitroprusside (SNP)-induced increase in mitochondrial reactive oxygen species (ROS) production and inhibiting the decrease of non-protein thiol content.

An exemplary neurotrophic agent that prevents the death of nerve cells and/or promotes regeneration of damaged brain tissue is AX-200. This drug prevents the death of nerve cells and promotes regeneration of damaged brain tissue. Sygnis Bioscience is evaluating the potential of the drug for the treatment of ALS.

An exemplary anti-inflammatory agent, cytokine modulator, and agent that reduce the level of activation of microglial cells is phosphatidylglycerol (PG)-containing liposomes, such as VP-025. VP-025 is in phase I trials at Vasogen for the treatment of ALS. Preclinical research has shown that VP-025 crosses the blood-brain barrier, producing potent anti-inflammatory activity, including cytokine modulation, by reducing the level of activation of microglial cells. This activity and evidence of a neuroprotective effect results in the preservation of function of specific neural pathways associated with memory and learning.

An exemplary cannabinoid CB1 receptor ligand, non-steroidal anti-inflammatory drug, and cannabinoid CB2 receptor ligand is Cannabinol. Such compounds may have neuroprotective effects against a variety of inflammatory, ischemic, and/or excitotoxic conditions.

An exemplary anti-oxidant and neuroprotective agent is (+)-R-Pramipexole. (+)-R-pramipexole, an inactive stereoisomer of the dopamine receptor agonist pramipexole hydrochloride, is currently undergoing phase II trials at the University of Virginia for the treatment of ALS. Previous studies have found that (+)-R-pramipexole may scavenge reactive oxygen species (ROS) and accumulate in mitochondria. Preclinical models of neural cell death caused by oxidative stress indicate that the drug induces neuroprotective effects.

An exemplary agent that encodes a ciliary neurotrophic factor is E1-Deleted recombinant Ad5 adenovirus encoding human CTNF (ciliary neurotrophic factor). Ciliary neurotrophic factors may improve neurite outgrowth, maintain neuronal structural integrity, regulate neuronal differentiation, and/or improve neuronal survival.

An exemplary agent that encodes a glial derived neurotrophic factor is E1-Deleted recombinant Ad5 adenovirus encoding human GDNF (glial derived neurotrophic factor). Glial derived neurotrophic factors may improve neurite outgrowth, maintain neuronal structural integrity, regulate neuronal differentiation, and/or improve neuronal survival. Agents that protect neurons from death, induce neurite outgrowth, and/or induce neurogenesis may be therapeutically useful in delaying neuron loss and/or stimulating the development of new neurons.

An exemplary an agent that encode neurotrophin 3 is E1-Deleted recombinant Ad5 adenovirus encoding human NT3 (NTF3) (neurotrophin 3). Neurotrophin 3 may improve neurite outgrowth, maintain neuronal structural integrity, regulate neuronal differentiation, and/or improve neuronal survival. Agents that protect neurons from death may be therapeutically useful in delaying neuron loss and/or stimulating the development of new neurons.

Another exemplary compound for use in a second therapy of the invention is Cholest-4-en-3-one oxime, such as TRO-19622. Phase I clinical trials are under way at Trophos for the treatment of ALS. TRO-19622 promotes motor neuron survival in culture and may reduce spinal motor neuron cell death in ALS patients. TRO-19622 is thought to act through stabilization of mitochondrial permeability transition pores and inhibition of pro-apoptotic factors.

Another exemplary compound for use in a second therapy of the invention is Thalidomide. Thalidomide has anti-angiogenic and immunomodulatory properties.

Another exemplary compound for use in a second therapy of the invention is Ceftriaxone. Ceftriaxone has anti-excitatory as well as anti-oxidant properties.

An exemplary free radical scavenger is MCI-186 (edaravone).

Other exemplary compounds for use in a second therapy of the invention include any compounds that are known or expected to improve, stabilize, eliminate, delay, or prevent ALS.

### Exemplary Formulations

One or several compounds described herein can be used in the preparation of a formulation, such as a pharmaceutical formulation, by combining the compound or compounds as an active ingredient with a pharmacologically acceptable carrier, which are known in the art. Depending on the therapeutic form of the system (e.g., transdermal patch vs. oral tablet), the carrier may be in various forms. In addition, pharmaceutical preparations may contain preservatives, solubilizers, stabilizers, re-wetting agents, emulgators, sweeteners, dyes, adjusters, salts for the adjustment of osmotic pressure, buffers, coating agents or antioxidants. Preparations comprising the compound, such as dimebon, may also contain other substances which have valuable therapeutic properties. Therapeutic forms may be represented by a usual standard dose and may be prepared by a known pharmaceutical method. Suitable formulations can be found, e.g., in Remington's Pharmaceutical Sciences, Mack Publishing Company, Philadelphia, Pa., 20^{th} ed. (2000), which is incorporated herein by reference.

### Exemplary Dosing Regimes

For use herein, unless clearly indicated otherwise, a compound or combination therapy of the invention may be administered to the individual by any available dosage form. In one variation, the compound or combination therapy is administered to the individual as a conventional immediate release dosage form. In one variation, the compound or combination therapy is administered to the individual as a sustained release form or part of a sustained release system, such as a system capable of sustaining the rate of delivery of a compound to an individual for a desired duration, which may be an extended duration such as a duration that is longer than the time required for a corresponding immediate-release dosage form to release the same amount (e.g., by weight or by moles) of compound or combination therapy, and can be hours or days. A desired duration may be at least the drug elimination half life of the administered compound or combination therapy and may be about any of, e.g., at least about 6 hours or at least about 12 hours or at least about 24 hours or at least about 30 hours or at least about 48 hours or at least about 72 hours or at least about 96 hours or at least about 120 hours or at least about 144 or more hours, and can be at least about one week, at least about 2 weeks, at least about 3 weeks, at least about 4 weeks, at least about 8 weeks, or at least about 16 weeks or more.

The compound or combination therapy may be formulated for any available delivery route, whether immediate or sustained release, including an oral, mucosal (e.g., nasal, sublingual, vaginal, buccal or rectal), parenteral (e.g., intramuscular, subcutaneous, or intravenous), topical or transdermal delivery form. A compound or combination therapy may be formulated with suitable carriers to provide delivery forms, which may be but are not required to be sustained release forms, that include, but are not limited to: tablets, caplets, capsules (such as hard gelatin capsules and soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

The amount of compound, such as dimebon, in a delivery form may be any effective amount, which may be from about 10 ng to about 1,500 mg or more of the single active ingredient compound of a monotherapy or of more than one active ingredient compound of a combination therapy. In one variation, a delivery form, such as a sustained release system, comprises less than about 30 mg of compound. In one variation, a delivery form, such as a single sustained release system capable of multi-day administration, comprises an amount of compound such that the daily dose of compound is less than about 30 mg of compound.

A treatment regimen involving a dosage form of compound, whether immediate release or a sustained release system, may involve administering the compound to the individual in dose of between about 0.1 and about 10 mg/kg of body weight, at least once a day and during the period of time required to achieve the therapeutic effect. In other variations, the daily dose (or other dosage frequency) of a hydrogenated pyrido[4,3-b]indole as described herein is between about 0.1 and about 8 mg/kg; or between about 0.1 to about 6 mg/kg; or between about 0.1 and about 4 mg/kg; or between about 0.1 and about 2 mg/kg; or between about 0.1 and about 1 mg/kg; or between about 0.5 and about 10 mg/kg; or between about 1 and about 10 mg/kg; or between about 2 and about 10 mg/kg; or between about 4 to about 10 mg/kg; or between about 6 to about 10 mg/kg; or between about 8 to about 10 mg/kg; or between about 0.1 and about 5 mg/kg; or between about 0.1 and about 4 mg/kg; or between about 0.5 and about 5 mg/kg; or between about 1 and about 5 mg/kg; or between about 1 and about 4 mg/kg; or between about 2 and about 4 mg/kg; or between about 1 and about 3 mg/kg; or between about 1.5 and about 3 mg/kg; or between about 2 and about 3 mg/kg; or between about 0.01 and about 10 mg/kg; or between about 0.01 and 4 mg/kg; or between about 0.01 mg/kg and 2 mg/kg; or between about 0.05 and 10 mg/kg; or between about 0.05 and 8 mg/kg; or between about 0.05 and 4 mg/kg; or between about 0.05 and 4 mg/kg; or between about 0.05 and about 3 mg/kg; or between about 10 kg to about 50 kg; or between about 10 to about 100 mg/kg or between about 10 to about 250 mg/kg; or between about 50 to about 100 mg/kg or between about 50 and 200 mg/kg; or between about 100 and about 200 mg/kg or between about 200 and about 500 mg/kg; or a dosage over about 100 mg/kg; or a dosage over about 500 mg/kg. In some embodiments, a daily dosage of dimebon is administered, such as a daily dosage that is less than about 0.1 mg/kg, which may include but is not limited to, a daily dosage of about 0.05 mg/kg.

The compound, such as dimebon, may be administered to an individual in accordance with an effective dosing regimen for a desired period of time or duration, such as at least about one month, at least about 2 months, at least about 3 months, at least about 6 months, or at least about 12 months or longer. In one variation, the compound is administered on a daily or intermittent schedule for the duration of the individual's life.

The dosing frequency can be about a once weekly dosing. The dosing frequency can be about a once daily dosing. The dosing frequency can be more than about once weekly dosing. The dosing frequency can be less than three times a day dosing. The dosing frequency can be about three times a week dosing. The dosing frequency can be about a four times a week dosing. The dosing frequency can be about a two times a week dosing. The dosing frequency can be more than about once weekly dosing but less than about daily dosing. The dosing frequency can be about a once monthly dosing. The dosing frequency can be about a twice weekly dosing. The dosing frequency can be more than about once monthly dosing but less than about once weekly dosing. The dosing frequency can be intermittent (e.g., once daily dosing for 7 days followed by no doses for 7 days, repeated for any 14 day time period, such as about 2 months, about 4 months, about 6 months or more). The dosing frequency can be continuous (e.g., once weekly dosing for continuous weeks). Any of the dosing frequencies can employ any of the compounds described herein together with any of the dosages described herein, for example, the dosing frequency can be a once daily dosage of less than 0.1 mg/kg or less than about 0.05 mg/kg of dimebon.

In one variation, dimebon is administered in a dose of 5 mg once a day. In one variation, dimebon is administered in a dose of 5 mg twice a day. In one variation, dimebon is administered in a dose of 5 mg three times a day. In one variation, dimebon is administered in a dose of 10 mg once a day. In one variation, dimebon is administered in a dose of 10 mg twice a day. In one variation, dimebon is administered in a dose of 10 mg three times a day. In one variation, dimebon is administered in a dose of 20 mg once a day. In one variation, dimebon is administered in a dose of 20 mg twice a day. In one variation, dimebon is administered in a dose of 20 mg three times a day. In one variation, dimebon is administered in a dose of 40 mg once a day. In one variation, dimebon is administered in a dose of 40 mg twice a day. In one variation, dimebon is administered in a dose of 40 mg three times a day.

### Exemplary Kits

The invention further provides kits comprising one or more compounds as described herein. The kits may employ any of the compounds disclosed herein and instructions for use. In one variation, the kit employs dimebon. The compound may be formulated in any acceptable form. The kits may be used for any one or more of the uses described herein, and, accordingly, may contain instructions for any one or more of the stated uses (e.g., treating and/or preventing and/or delaying the onset and/or the development of ALS).

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may optionally include a set of instructions, generally written instructions, although electronic storage media (e.g., magnetic diskette or optical disk) containing instructions are also acceptable, relating to the use of component(s) of the methods of the present invention (e.g., treating, preventing and/or delaying the onset and/or the development of ALS). The instructions included with the kit generally include information as to the components and their administration to an individual.

The following Examples are provided to illustrate but not limit the invention.

### Examples

### Example 1

### Determination of Toxicity Properties of Dimebon

Dimebon, 2,8-dimethyl-5-(2-(6-methyl-3-pyridyl)-ethyl)-2,3,4,5-tetrahydro-1H-pyrido(4,3-b)indol dihydrochloride, was used as a representative compound of hydrogenated pyrido(4,3-b)indoles.

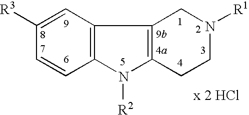

where R¹ and R³ are methyls, and

R² is 2-(6-methyl-3-pyridyl)-ethyl

Dimebon was evaluated for toxicity levels in wildtype Drosophila fruit flies as described in U.S. Provisional Patent Application No. 60/723,403. Dimebon was administered daily at doses ranging from 10 μM to 1 mM to explore its toxicity. An untreated control group was also studied in this experiment. The concentrations given were concentrations of dimebon in the food that animals drink/eat ad libitum. The food consisted of cornmeal, dextrose, yeast and agar.

About 500 wild type Drosophila eggs were collected on grape juice plates, washed with distilled water and transferred 100 per vial to grow at 25 degrees C. The adult progeny were scored after eclosing (emerging from the pupal case) beginning 10 days later. The criteria used for toxicity were the number (%) of animals that eclose and the time of the eclosing. For example, fewer animals may emerge from the pupal case if a drug is toxic or the same number of animals may eclose but more slowly than the untreated control group.

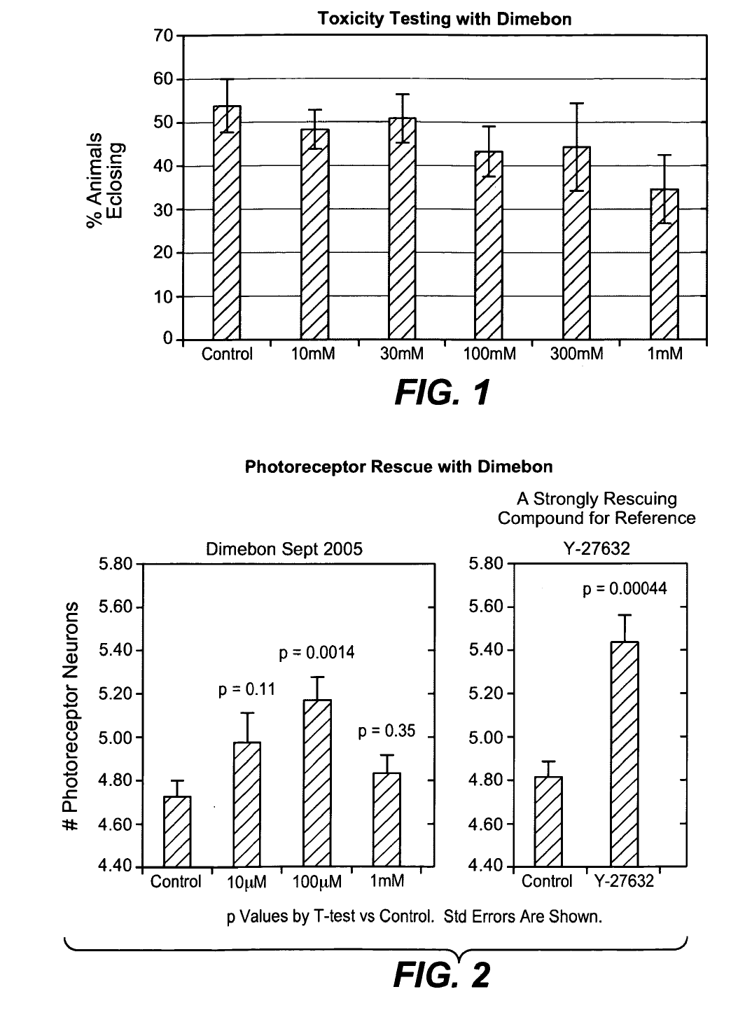

As illustrated in FIG. 1, dimebon caused no significant toxicity until a dose of 1 mM was reached, at which point there was a decrease in the % of animals eclosing and the timing of emergence was slowed by approximately 1 day.

### Example 2

### Determination of Dimebon's Ability to Inhibit Huntingtin-Induced Neurodegeneration of Photoreceptor Neurons in Drosophila Eyes

As discussed in U.S. application Ser. No. 60/723,403 and further below, it has been discovered that dimebon, a representative member of a class of compounds disclosed herein, had strikingly positive results in the art-accepted Drosophila model of Huntington's disease, and exhibited enhanced protective effects when compared to a control. This result supports the ability of the hydrogenated pyrido[4,3-b]indoles described herein to inhibit neuronal cell death, which is a characteristic of ALS.

The Drosophila fruit fly is considered an excellent choice for modeling neurodegenerative diseases because it contains a fully functional nervous system with an architecture that separates specialized functions such as vision, smell, learning and memory in a manner not unlike that of mammalian nervous systems. Furthermore, the compound eye of the fruit fly is made up of hundreds of repeating constellations of specialized neurons which can be directly visualized through a microscope and upon which the ability of potential neuroprotective drugs to directly block neuronal cell death can easily be assessed. Finally, among human genes known to be associated with disease, approximately 75% have a Drosophila fruit fly counterpart.

In particular, the expression of mutant huntintin protein in Drosophila fruit flies results in a fly phenotype that exhibits some of the features of human Huntington's disease. First, the presumed etiologic agent in Huntington's disease (mutant huntingtin protein) is encoded by a repeated triplet of nucleotides (CAG) which are called polyglutamine or polyQ repeats. In humans, the severity of Huntington's disease is correlated with the length of polyQ repeats. The same polyQ length dependency is seen in Drosophila. Secondly, no neurodegeneration is seen at early ages (early larval stages) in flies expressing the mutant huntingtin protein, although at later life stages (mature larval, pupal and aging adult stages), flies do develop the disease, similarly to humans, who generally manifest the first signs and symptoms of Huntington's disease starting in the fourth and fifth decades of life. Third, the neurodegeneration seen in flies expressing the mutant huntingtin gene is progressive, as it is in human patients with Huntington's disease. Fourth, the neuropathology in huntingtin-expressing flies leads to a loss of motor function as it does in similarly afflicted human patients. Last, flies expressing the mutant huntingtin protein die an early death, as do patients with Huntington's disease. For these reasons, compounds which show a neuroprotective effect in the Drosophila model of Huntington's disease are expected to be the most likely compounds to have a beneficial effect in humans.

Dimebon, 2,8-dimethyl-5-(2-(6-methyl-3-pyridyl)-ethyl)-2,3,4,5-tetrahydro-1H-pyrido(4,3-b)indol dihydrochloride, was used as a representative compound of (4,3-b)indoles.

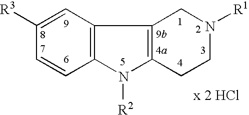

where R¹ and R³ are methyls, and

R² is 2-(6-methyl-3-pyridyl)-ethyl

Dimebon was administered to one group of transgenic Drosophila engineered to express the mutant huntingtin protein in all their neurons as described in U.S. Provisional Patent Application No. 60/723,403. This was accomplished by cloning a foreign gene into transposable p-element DNA vectors under control of a yeast upstream activator sequence that was activated by the yeast GAL4 transcription factor. These promoter fusions were injected into fly embryos to produce transgenic animals. The foreign gene is silent until crossed to another transgenic strain of flies expressing the GAL4 gene in a tissue specific manner. The Elav>Gal4 which expresses the transgene in all neurons from birth until death was used in the experiments described.

The two types of transgenic animals were crossed in order to collect enough closely matched aged controls to study. The crossed aged-matched adults (−20 per dosing group) were placed on drug containing food for 7 days. Animals were transferred to fresh food daily to minimize any effects caused by instability of the compounds. Survival was scored daily. At day 7, animals were sacrificed and the number of photoreceptor neurons surviving was counted. Scoring was by the pseudopupil method where individual functioning photoreceptors are revealed by light focused on the back of the head and visualized as focused points of light under a compound microscope focused at the photoreceptor level of the eye. Dimebon was found to protect photoreceptors in a dose-dependant manner.

As shown in FIG. 2, when tested for its ability to inhibit mutant huntingtin-induced neurodegeneration of photoreceptor neurons in Drosophila eyes (which are reflective of neurodegenerative changes in fly brains), dimebon at a dose of 100 μM caused a statistically significant (p=0.0014) rescue of neurons compared to the untreated controls. The magnitude of effect seen is comparable to a historical positive control, Y-27632, a small molecule rho kinase inhibitor considered to be a strongly rescuing reference compound. A dose-dependent rescue of fly neurons was observed with dimebon, with a lesser but still apparent rescue of neurons observed at the 10 μM dose compared to the 100 μM dose. The 1 mM dimebon dose (established in the previous toxicity study to be a somewhat toxic dose) still appeared to cause neuronal rescue, but to a lesser extent than the 100 μM or 10 μM dimebon doses.

The presented results suggest that dimebon statistically reliably inhibits mutant huntingtin-induced neurodegeneration of neurons in Drosophila eyes. Results in the described Drosophila model historically have correlated very well with transgenic mouse models for Huntington's disease. The close resemblance of the Drosophila model to the human Huntington's disease condition is described in J. L. Marsh et al., “Fly models of Huntington's Disease,” Human Molecular Genetics, 2003, vol 12, review issue 2, R187-R193. Thus, dimebon is believed to be a promising new agent for use in medicine to treat, prevent, slow the progression or delay the onset and/or development of Huntington's disease. All of the above suggest that dimebon and the class of compounds disclosed herein are promising effective agents for the treatment, prevention, slowing the progression of or delaying the onset and/or development of Huntington's disease.

### Example 3

### Use of an in vitro Model to Determine the Ability to Compounds of the Invention to Treat, Prevent and/or Delay the Onset and/or the Development of Amyotrophic Lateral Sclerosis

In vitro models of ALS can be used to determine the ability of any of the hydrogenated pyrido[4,3-b]indoles (such as dimebon) or combination therapies described herein to reduce cell toxicity that is induced by a SOD1 mutation. A reduction in cell toxicity is indicative of the ability to treat, prevent and/or delay the onset and/or the development of ALS in mammals, such as humans.

In one exemplary in vitro model of ALS, N2a cells (e.g., the mouse neuroblastoma cell cline N2a sold b y InPro Biotechnology, South San Francisco, Calif., USA) are transiently transfected with a mutant SOD1 in the presence or absence of various concentrations of a hydrogenated pyrido[4,3-b]indole, such as dimebon. Standard methods can be used for this transfection, such as those described by Y. Wang et al., (Journal of Nuclear Medicine, 46(4):667-674, 2005). Cell toxicity can be measured using any routine method, such as cell counting, immunostaining, and/or MTT assays to determine whether the hydrogenated pyrido[4,3-b]indole attenuates mutant SOD1-mediated toxicity in N2a cells (see, for example, U.S. Pat. No. 7,030,126; Y. Zhang et al., Proc. Natl. Acad. Sci. USA, 99(11):7408-7413, 2002; or S. Fernaeus et al., Neurosci Lett. 389(3):133-6, 2005).

### Example 4

### Use of an in vivo Model to Determine the Ability to Compounds of the Invention to Treat, Prevent and/or Delay the Onset and/or the Development of Amyotrophic Lateral Sclerosis

In vivo models of ALS can also be used to determine the ability of any of the hydrogenated pyrido[4,3-b]indoles (such as dimebon) or combination therapies described herein to treat, prevent and/or delay the onset and/or the development of ALS in mammals, such as humans. Several animal models of ALS or motor neuron degeneration have been developed by others, such as those described in U.S. Pat. No. 7,030,126 and U.S. Pat. No. 6,723,315.

For example, several lines of transgenic mice expressing mutated forms of SOD responsible for the familial forms of ALS have been constructed as murine models of ALS (U.S. Pat. No. 6,723,315). Transgenic mice overexpressing mutated human SOD carrying a substitution of glycine 93 by alanine (FALS_{G93A} mice) have a progressive motor neuron degeneration expressing itself by a paralysis of the limbs, and die at the age of 4-6 months (Gurney et al., Science, 264, 1772-1775, 1994). The first clinical signs consist of a trembling of the limbs at approximately 90 days, then a reduction in the length of the step at 125 days. At the histological level, vacuoles of mitochondrial origin can be observed in the motor neurons from approximately 37 days, and a motor neurons loss can be observed from 90 days. Attacks on the myelinated axons are observed principally in the ventral marrow and a little in the dorsal region. Compensatory collateral reinnervation phenomena are observed at the level of the motor plaques.

FALS_{G93A} mice constitute a very good animal model for the study of the physiopathological mechanisms of ALS as well as for the development of therapeutic strategies. These mice exhibit a large number of histopathological and electromyographic characteristics of ALS. The electromyographic performances of the FALS_{G93A} mice indicate that they fulfill many of the criteria for ALS: (1) reduction in the number of motor units with a concomitant collateral reinnervation, (2) presence of spontaneous denervation activity (fibrillations) and of fasciculation in the hind and fore limbs, (3) modification of the speed of motor conduction correlated with a reduction in the motor response evoked, and (4) no sensory attack. Moreover, the facial nerve attacks are rare, even in the aged FALS_{G93A} mice, which is also the case in patients. The FALS_{G93A} mice are available from Transgenic Alliance (L'Arbresle, France). Additionally, heterozygous transgenic mice carrying the human SOD1 (G93A) gene can be obtained from Jackson Laboratory (Bar Harbor, Me., USA) (U.S. Pat. No. 7,030,126). These mice have 25 copies of the human G93A SOD mutation that are driven by the endogenous promoter. Survival in the mouse is copy dependent. Mouse heterozygotes developing the disease can be identified by PCR after taking a piece of tail and extracting DNA.

Other animal models having motor neurons degeneration exist (U.S. Pat. No. 6,723,315; Sillevis-Smitt & De Jong, J. Neurol. Sci., 91, 231-258, 1989; Price et al., Neurobiol. Disease, 1, 3-11, 11994), either following an acute neurotoxic lesion (treatment with IDPN, with excitotoxins) or due to a genetic fault (wobbler, pmn, Mnd mice or HCSMA Dog). Among the genetic models, the pmn mice are particularly well characterized on the clinical, histological and electromyographic level. The pmn mutation is transmitted in the autosomal recessive mode and has been localized on chromosome 13. The homozygous pmn mice develop a muscular atrophy and paralysis which is manifested in the rear members from the age of two to three weeks. All the non-treated pmn mice die before six to seven weeks of age. The degeneration of their motor neurones begins at the level of the nerve endings and ends in a massive loss of myelinized fibres in the motor nerves and especially in the phrenic nerve which ensures the inervation of the diaphragm. Contrary to the FALS_{G93A} mouse, this muscular denervation is very rapid and is virtually unaccompanied by signs of reinervation by regrowth of axonal collaterals. On the electromyographic level, the process of muscular denervation is characterized by the appearance of fibrillations and by a significant reduction in the amplitude of the muscular response caused after supramaximal electric stimulation of the nerve.

A line of Xt/pmn transgenic mice has also been used previously as another murine model of ALS (U.S. Pat. No. 6,723,315). These mice are obtained by a first crossing between C57/B156 or DBA2 female mice and Xt pmn⁺/Xt⁺pmn male mice (strain 129), followed by a second between descendants Xt pmn⁺/Xt⁺pmn⁺ heterozygous females (N1) with initial males. Among the descendant mice (N2), the Xt pmn⁺/Xt⁺ pmn double heterozygotes (called “Xt pmn mice”) carrying an Xt allele (demonstrated by the Extra digit phenotype) and a pan allele (determined by PCR) were chosen for the future crossings.

In one exemplary method for testing the activity of one or more hydrogenated pyrido[4,3-b]indoles described herein in an in vivo model of ALS, female mice (B6SJL) are purchased to breed with the transgenic males that overexpress a mutated SOD carrying a substitution of glycine 93 by alanine (e.g., FALS_{G93A} mice). Two females are put in each cage with one male and monitored at least daily for pregnancy. As each pregnant female is identified, it is removed from the cage and a new non-pregnant female is added. Since 40-50% of the pups are expected to be transgenic, a colony of, for example, at least 200 pups can be born at approximately the same time. After genotyping at three weeks of age, the transgenic pups are weaned and separated into different cages by sex.

At least 80 transgenic mice (both male and female) are randomized into four groups: 1) vehicle treated (20 mice), 2) dose 1 (3 mg/kg/day; 20 mice), 3) dose 2 (10 mg/kg/day; 20 mice) and 3) dose 3 (30 mg/kg/day; 20 mice). Mice are evaluated daily. This evaluation includes analysis of weight, appearance (fur coat, activities, etc.) and motor coordination. Treatment starts at approximate stage 3 and continues until mice are euthanized. The hydrogenated pyrido[4,3-b]indole being tested is administered to the mice in their food.

The onset of clinical disease is scored by examining the mouse for tremor of its limbs and for muscle strength. The mice are lifted gently by the base of the tail and any muscle tremors are noted, and the hind limb extension is measured. Muscle weakness is reflected in the inability of the mouse to extend its hind limbs. The mice are scored on a five point scale for symptoms of motor neuron dysfunction: 5—no symptoms; 4—weakness in one or mote limbs; 3—limping in one or more limbs; 2—paralysis in one or more limbs; 1—animal negative for reflexes, unable to right itself when placed on its back.

In animals showing signs of paralysis, moistened food pellets are placed inside the cage. When the mice are unable to reach food pellets, nutritional supplements are administered through assisted feeding (Ensure, p.o, twice daily). Normal saline is supplemented by i.p. administration, 1 ml twice daily if necessary. In addition, these mice are weighed daily. If necessary, mice are cleaned by the research personnel, and the cage bedding is changed frequently. At end-stage disease, mice lay on their sides in their cage. Mice are euthanized immediately if they cannot right themselves within 10 seconds or if they lose 20% of their body weight.

Spinal cords are collected from the fourth, eighth, twelfth, sixteenth and twentieth animal euthanized in each treatment group (total of five animals per treatment group, twenty animals total). These spinal cords are analyzed for mutant SOD1 content in mitochondria using standard methods (see, for example, J. Liu et al., Neuron, 43(1):5-17, 2004).

If desired, the effect of the hydrogenated pyrido[4,3-b]indole in the ALS mouse model can be further characterized using standard methods to measure the size of the bicep muscles, the muscle morphology, the muscle response to electric stimulation, the number of spinal motor neurons, muscle function, and/or the amount of oxidative damage, e.g., as described in U.S. Pat. No. 6,933,310 or U.S. Pat. No. 6,723,315.

Compounds that result in less muscle weakness and/or a smaller reduction in the number of motor neurons compared to the vehicle control in any of the above in vivo models of ALS are expected to be the most likely compounds to have a beneficial effect in humans for the treatment or prevention of ALS.

### Example 5

### Evaluation of Dimebon in a G93AmSOD Transgenic Mouse Treatment Model

A G93AmSOD transgenic mouse treatment model (see, or example, Gurney M E et al., 1994. Science 264 1772-1775) can be used to determine the ability of any of the hydrogenated pyrido[4,3-b]indoles (such as dimebon) or combination therapies described herein to treat ALS in mammals. Dimebon, 2,8-dimethyl-5-(2-(6-methyl-3-pyridyl)-ethyl)-2,3,4,5-tetrahydro-1H-pyrido(4,3-b)indol dihydrochloride, was used as a representative compound of (4,3-b)indoles.

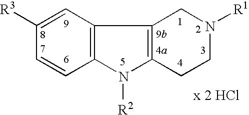

where R¹ and R³ are methyls, and

R² is 2-(6-methyl-3-pyridyl)-ethyl

For this study, G93AmSOD mice were randomized into 4 treatment groups. Mice were weaned and raised on a normal diet for 85 days. Beginning at approximately day 80 or earlier if noted clinically, animals underwent daily assessment for hind limb weakness (time to stage 3 disease). In general, this occurred within a week of day 85. At 85 days, mice were given dimebon in the drinking water at the following concentrations: vehicle control (0 mg/kg/day), low dose (3 mg/kg/day), medium dose (10 mg/kg/day), and high dose (30 mg/kg/day). The drinking water was changed every 3-4 days, and each cage held approximately 3-5 animals.

Animals were weighed and analyzed daily to assess their strength and function. The day during which hind limb paralysis occurred was recorded (progression to stage 2 disease). Also recorded was the day at which the animals could no longer right themselves after 30 seconds (progression to stage 1 disease—a surrogate for mortality). Upon reaching stage 1 disease, animals were euthanized. When animals were found to have lost 10% of body weight, they were offered ensure hand feedings daily. When animals were no longer able to reliably reach the drinking water, they were given their daily mg/kg dose by intraperitoneal injection. Analyses were performed to compare the groups in terms of time to reach stage 2 and time to reach stage 1. As described further below, a Cox proportional hazards model including the effect of treatment group was fit.

### Time to Stage 2

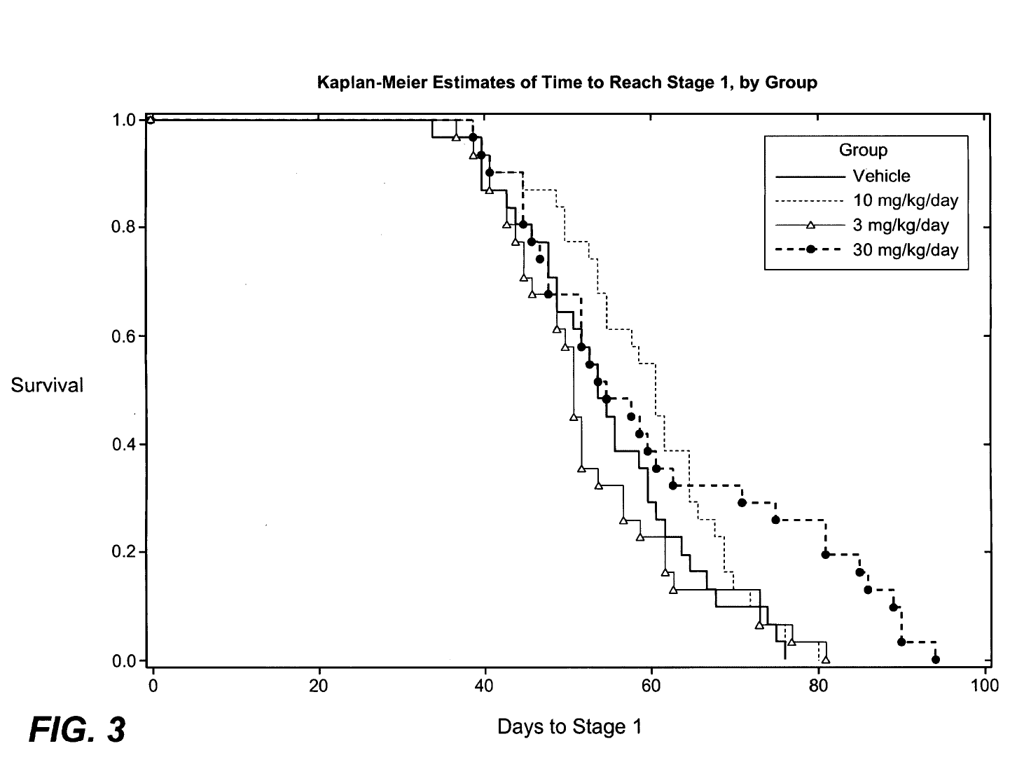

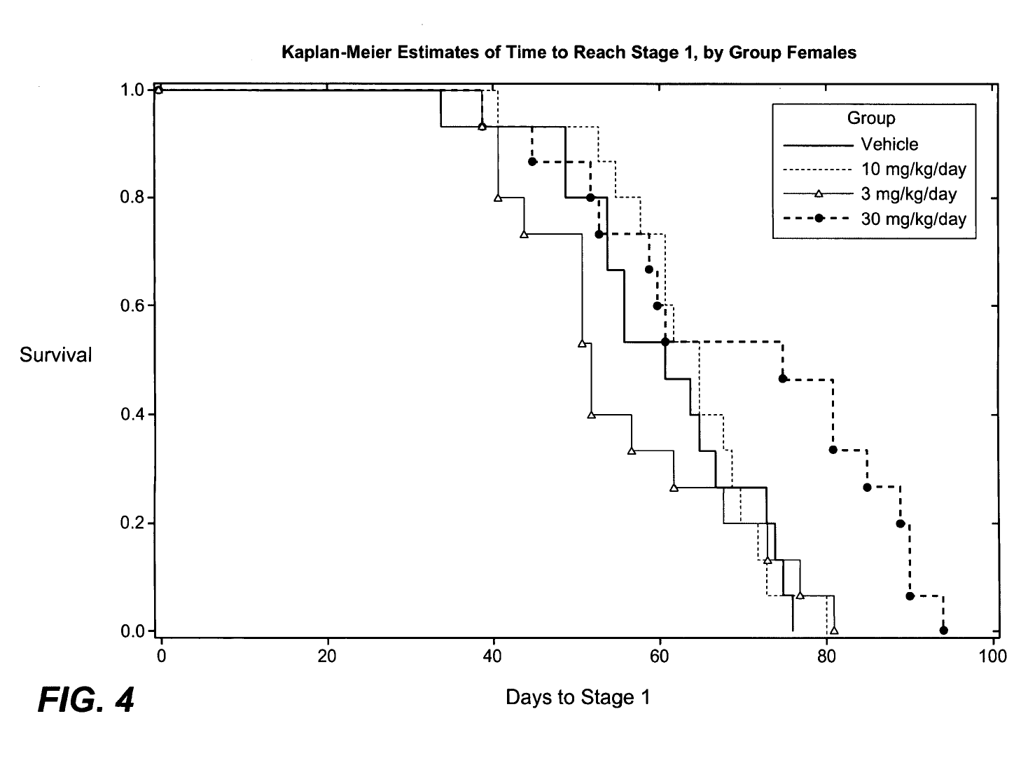

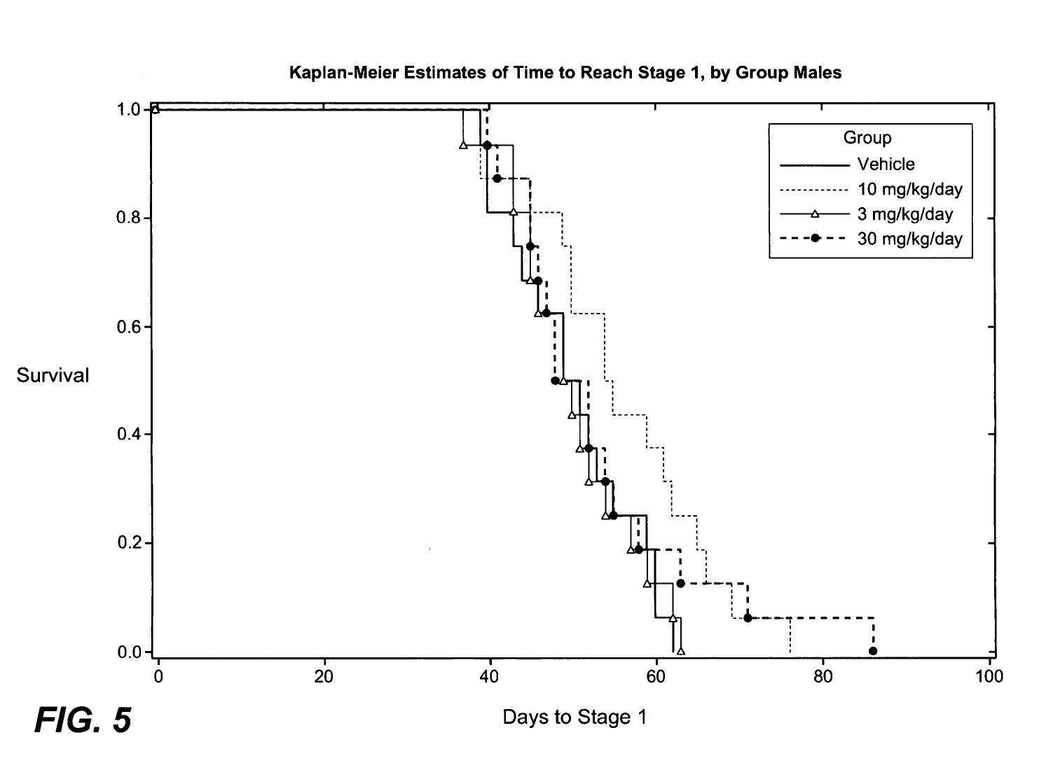

A Cox proportional hazards regression model including the effect of treatment group was fit for the time to stage 2 for both sexes combined. In addition to testing the null hypothesis of no difference among the four groups, pair wise comparisons of each of the treated groups to the control group were tested. The model was fit using the SAS PHREG procedure. The same type of model was then fit to the data for each sex separately. Table 1 and FIGS. 3-5 summarize the results from the three models.

TABLE 1
Time to Stage 2: Results from Cox Proportional
Hazards Regression Models

| | Both Sexes | | |
|---|---|---|---|
| Comparison | Combined | Females | Males |
| p-value from likelihood ratio test | 0.0405 | 0.0520 | 0.2670 |
| of no difference among groups | | | |
| 3 mg/kg/day versus vehicle: | | | |
| hazard ratio | 0.969 | 0.882 | 0.984 |
| p-value | 0.9025 | 0.7359 | 0.9629 |
| 10 mg/kg/day versus vehicle: | | | |
| hazard ratio | 0.652 | 0.659 | 0.545 |
| p-value | 0.0948 | 0.2585 | 0.0924 |
| 30 mg/kg/day versus vehicle: | | | |
| hazard ratio | 0.522 | 0.359 | 0.698 |
| p-value | 0.0182 | 0.0145 | 0.3301 |

In both sexes combined, the overall difference among the four groups was statistically significant. The difference was nearly significant in females. In all three analyses, the hazard ratios decreased monotonically as the dose increased. In both sexes combined and in females, the difference between the 30 mg/kg/day group and the vehicle group was statistically significant. Based on these results, Table 2 displays for each of the three treatment groups the group mean expressed as a percentage of the mean in the vehicle group.

TABLE 2
Mean Time to Stage 2 (Expressed as a Percentage of
the Mean in the Vehicle Group)

| | Both Sexes | | |
|---|---|---|---|
| Treatment Group | Combined | Females | Males |
| 3 mg/kg/day | 99.8% | 100.2% | 99.4% |
| 10 mg/kg/day | 104.9% | 104.9% | 104.9% |
| 30 mg/kg/day | 104.8% | 107.7% | 101.8% |

### Time to Stage 1

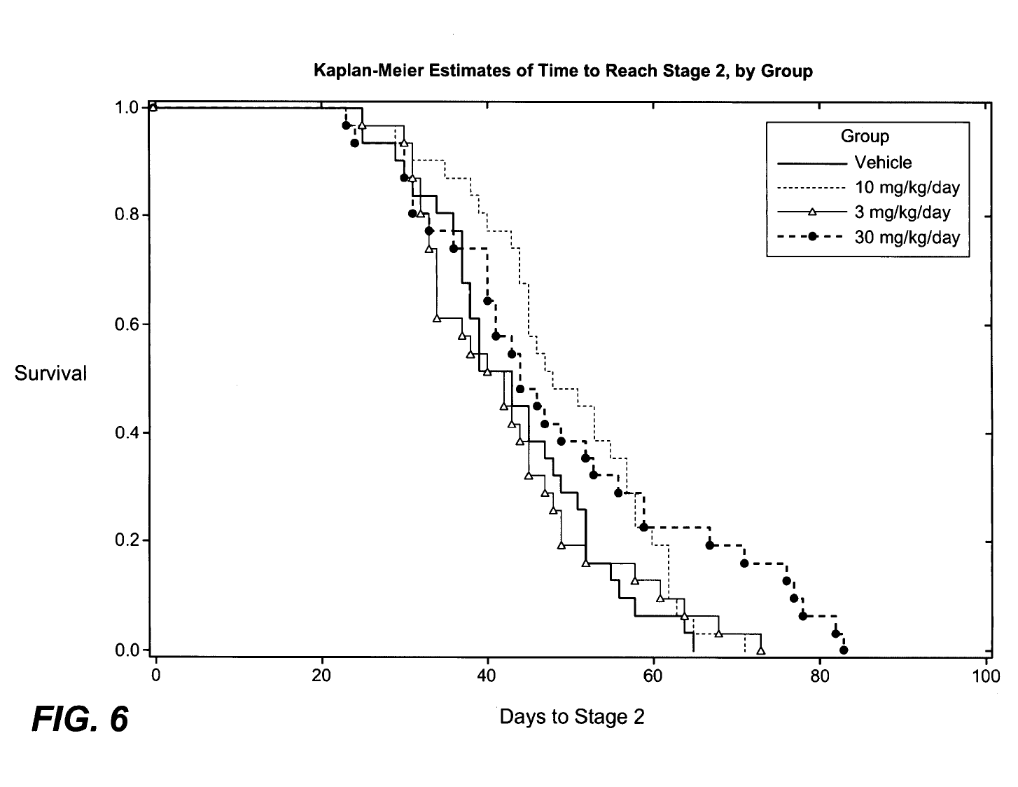

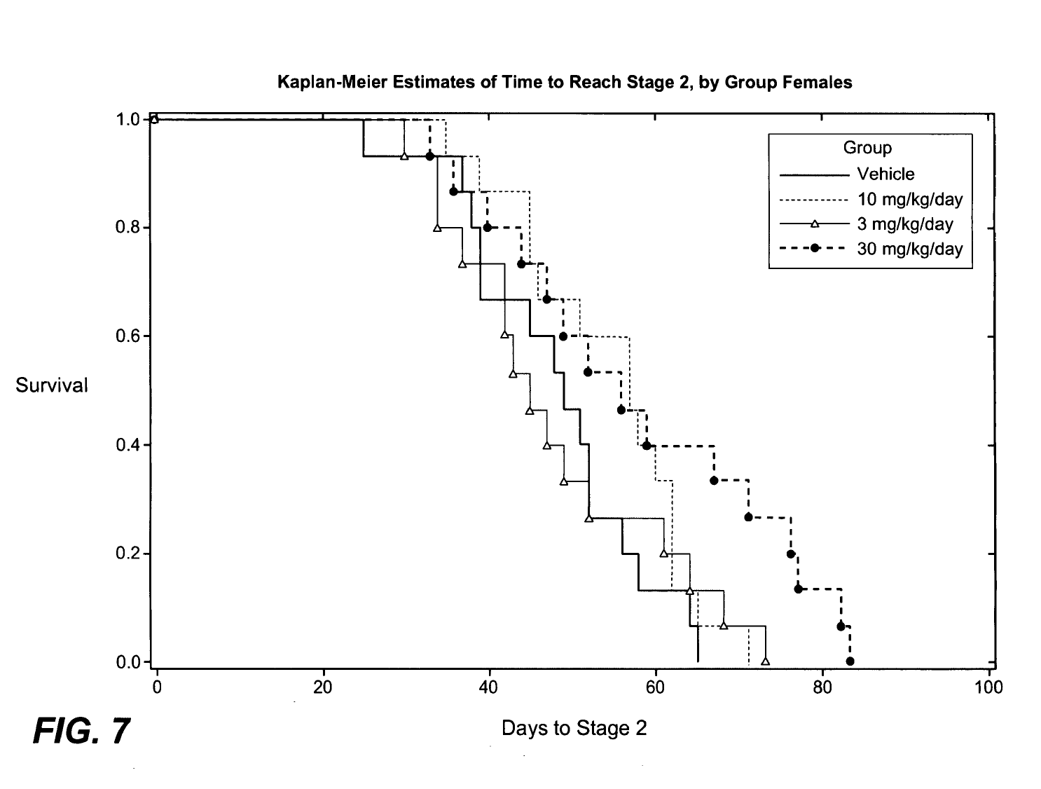

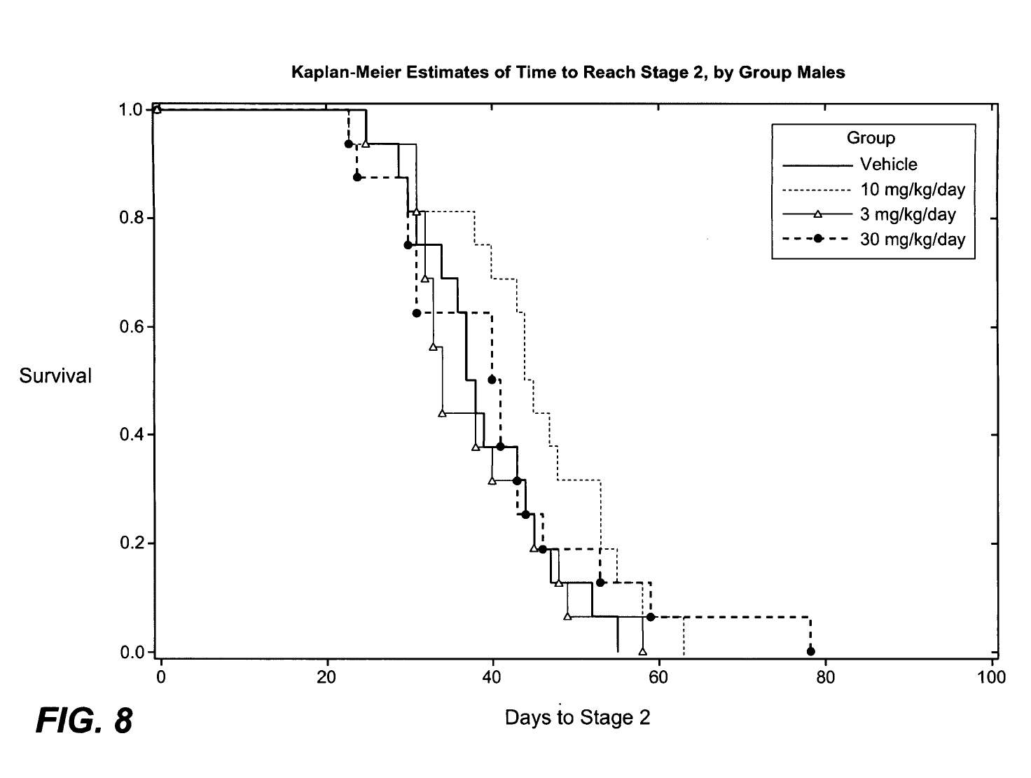

A Cox proportional hazards regression model including the effect of treatment group was fit for the time to stage 1 for both sexes combined. In addition to testing the null hypothesis of no difference among the four groups, pair wise comparisons of each of the treated groups to the control group were tested. The model was fit using the SAS PHREG procedure. The same type of model was then fit to the data for each sex separately. Table 3 and FIGS. 6-8 summarize the results from the three models.

TABLE 3
Time to Stage 1: Results from Cox Proportional
Hazards Regression Models

| | Both Sexes | | |
|---|---|---|---|
| Comparison | Combined | Females | Males |
| p-value from likelihood ratio test | 0.0182 | 0.0098 | 0.2283 |
| of no difference among groups | | | |
| 3 mg/kg/day versus vehicle: | | | |
| hazard ratio | 1.112 | 1.089 | 0.967 |
| p-value | 0.6783 | 0.8194 | 0.9253 |
| 10 mg/kg/day versus vehicle: | | | |
| hazard ratio | 0.738 | 0.855 | 0.524 |
| p-value | 0.2337 | 0.6698 | 0.0784 |
| 30 mg/kg/day versus vehicle: | | | |
| hazard ratio | 0.505 | 0.314 | 0.647 |
| p-value | 0.0151 | 0.0086 | 0.2439 |

In both sexes combined, as well as in females, the overall difference among the four groups was statistically significant. Although the hazard rate for the 3 mg/kg/day group versus vehicle was slightly larger than one in both sexes combined and in females, the magnitude of the increase was small. In both sexes combined and in females, the hazard ratio for the 30 mg/kg/day comparison was smaller than the corresponding hazard ratio for the 10 mg/kg/day comparison. However, in males, the smallest hazard ratio was for the 10 mg/kg/day comparison. In both sexes combined and in females, the difference between the 30 mg/kg/day group and the vehicle group was statistically significant. Based on these results, Table 4 displays for each of the three treatment groups the group mean expressed as a percentage of the mean in the vehicle group.

TABLE 4
Mean Time to Stage 1 (Expressed as a Percentage of
the Mean in the Vehicle Group)

| | Both Sexes | | |
|---|---|---|---|
| Treatment Group | Combined | Females | Males |
| 3 mg/kg/day | 98.8% | 97.4% | 100.2% |
| 10 mg/kg/day | 103.3% | 102.3% | 104.2% |
| 30 mg/kg/day | 104.7% | 107.0% | 102.4% |

In summary, for both sexes combined the overall difference in survival (time to reach stage 1) between group was statistically significant (p=0.04) and nearly reached statistical significance for female mice (p=0.052). In all three survival analyses, the hazard ratios decreased monotonically as the dose increased, suggesting a dose-response relationship to treatment effect. In both sexes combined and in females, the difference between the 30 mg/kg/day group and the vehicle control group was statistically significant (p=0.018-0.014). Similar findings were noted in analyses of disease progression (time to reach stage 2). No censoring of animals was required.

### Example 6

### Evaluation of Dimebon in a G93AmSOD Transgenic Mouse Prophylaxis Model

A G93AmSOD transgenic mouse prophylaxis model can be used to determine the ability of any of the hydrogenated pyrido[4,3-b]indoles (such as dimebon) or combination therapies described herein to prevent and/or delay the onset and/or the development of ALS in mammals. In this prophylaxis model, treatment starts on day 32 (before symptoms start) rather than day 85 (after symptoms start) as done for the treatment model in Example 5. Dimebon, 2,8-dimethyl-5-(2-(6-methyl-3-pyridyl)-ethyl)-2,3,4,5-tetrahydro-1H-pyrido(4,3-b)indol dihydrochloride, was used as a representative compound of (4,3-b)indoles.

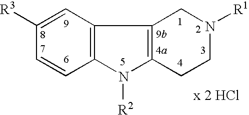

where R¹ and R³ are methyls, and

R² is 2-(6-methyl-3-pyridyl)-ethyl

For this study, approximately 108 G93AmSOD mice were randomized into 4 treatment groups. Mice were weaned and raised on normal diet for 32 days. Beginning at approximately day 80 or earlier if noted clinically, animals underwent daily assessment for hind limb weakness (time to stage 3 disease). At approximately 32 days, mice were given dimebon in the drinking water at the following concentrations: vehicle control (0 mg/kg/day), low dose (10 mg/kg/day), medium dose (30 mg/kg/day), and high dose (100 mg/kg/day). Drinking water was changed every 3-4 days, and each cage held approximately 3-5 animals.

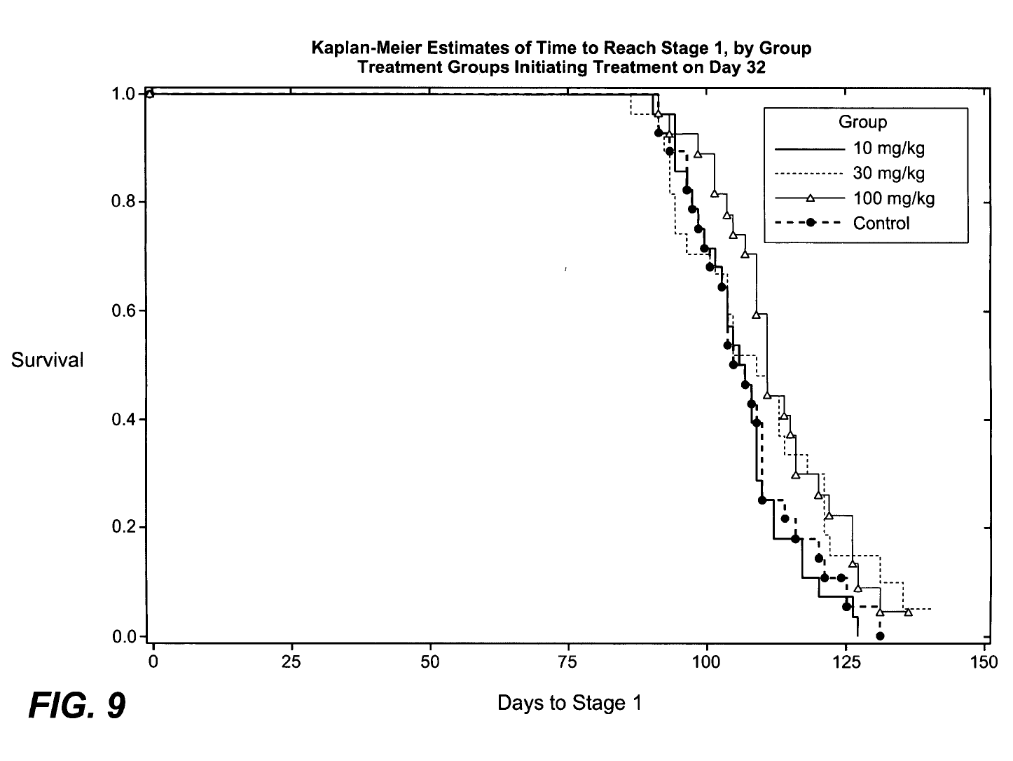

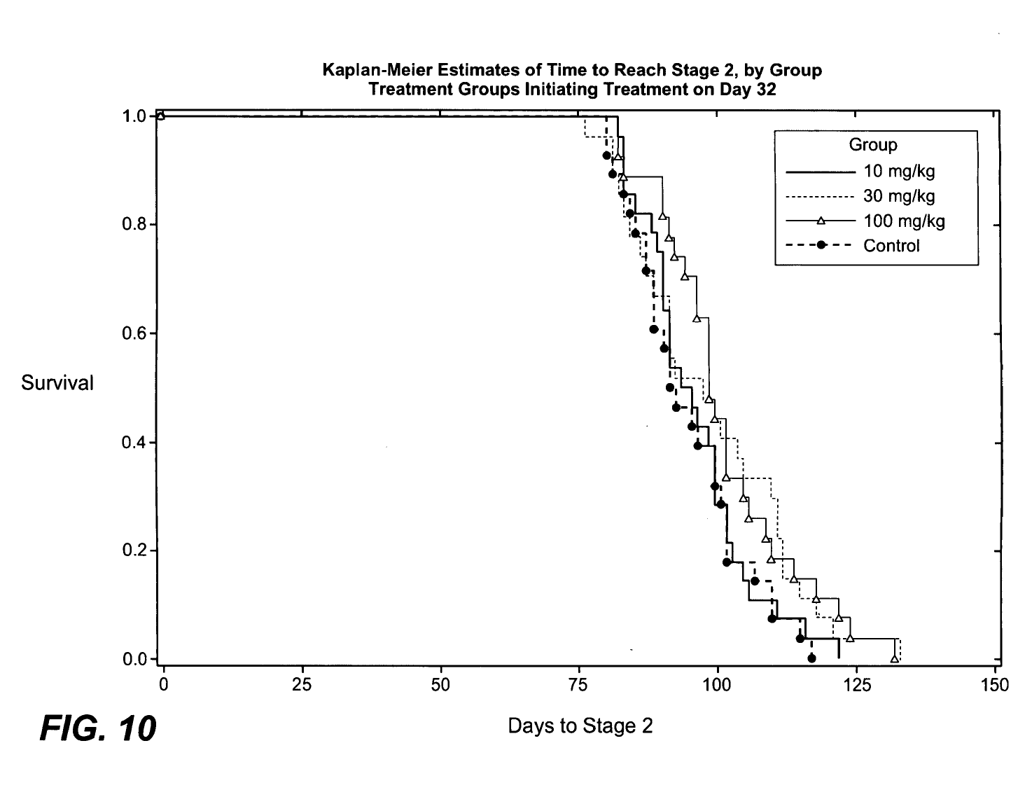

Animals were weighed and analyzed daily to assess their strength and function. The day during which hind limb paralysis occurred was recorded (progression to stage 2 disease). Also recorded was the day at which the animals could no longer right themselves after 30 seconds (progression to stage 1 disease—a surrogate for mortality). Upon reaching stage 1 disease, animals were euthanized. When animals were found to have lost 10% of body weight, they were offered ensure hand feedings daily. When animals were no longer able to reliably reach the drinking water, they were given their daily mg/kg dose by a single daily intraperitoneal injection. The groups were compared in terms of time to reach stage 3, time to reach stage 2, and time to reach stage 1 (FIGS. 9 and 10). These same analyses were repeated with the animals stratified by gender. Analytic methods were essentially the same as for Examples 5.

### Example 7

### Evaluation of a Higher Dose of Dimebon in a G93AmSOD Transgenic Mouse Treatment Model

If desired, a higher dose of dimebon can be tested in a G93AmSOD transgenic mouse treatment model to further characterize the ability of dimebon to treat ALS in mammals. For this study, dimebon, 2,8-dimethyl-5-(2-(6-methyl-3-pyridyl)-ethyl)-2,3,4,5-tetrahydro-1H-pyrido(4,3-b)indol dihydrochloride, was used as a representative compound of (4,3-b)indoles.

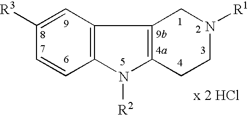

where R¹ and R³ are methyls, and

R² is 2-(6-methyl-3-pyridyl)-ethyl

This study was performed essentially as described for Example 5 except that approximately 30 animals were randomized into two groups: a vehicle control group (0 mg/kg/day) and a high dose group (100 mg/kg/day). The analytical methods used were essentially the same as those described in Examples 5 and 6. A comparison of the effects of early (day 32) versus late (day 85) treatment initiation was performed.

### Example 8

### Comparison of the Effect of a Combination of Riluzole and Dimebon to Riluzole Alone in a G93AmSOD Transgenic Mouse Prophylactic Model

If desired, a G93AmSOD transgenic mouse prophylaxis model can be used to determine the ability of any of the combination therapies described herein (e.g., a hydrogenated pyrido[4,3-b]indole such as dimebon and a second therapy) to prevent and/or delay the onset and/or the development of ALS in mammals. For this study, dimebon, 2,8-dimethyl-5-(2-(6-methyl-3-pyridyl)-ethyl)-2,3,4,5-tetrahydro-1H-pyrido(4,3-b)indol dihydrochloride, is being used as a representative compound of (4,3-b)indoles.

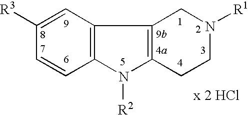

where R¹ and R³ are methyls, and

R² is 2-(6-methyl-3-pyridyl)-ethyl

Riluzole is being used as a representative second therapy that is useful for treating, preventing and/or delaying the onset and/or development of ALS.

This study is performed essentially as described for Example 6 except that approximately 60 animals are randomized into two groups. At approximately 32 days, mice are given dimebon and/or riluzole in the drinking water at the following concentrations:

Riluzole (30 mg/kg/day) and dimebon (30 mg/kg/day)

Riluzole (30 mg/kg/day) alone

Other aspects of care are essentially as described for Example 6. Animals are analyzed to determine the time required for them to reach stage 3, stage 2, and then stage 1. Clinical observations are made to assess for any signs of toxicity.

### Example 9

### Evaluation of the Effect of Dimebon on Motor Neuron Cells

If desired, a G93AmSOD transgenic mouse prophylaxis model can be used to determine the ability of any of the hydrogenated pyrido[4,3-b]indole (such as dimebon) or combination therapies described herein to affect the number of lower motor neurons. For this study, dimebon, 2,8-dimethyl-5-(2-(6-methyl-3-pyridyl)-ethyl)-2,3,4,5-tetrahydro-1H-pyrido(4,3-b)indol dihydrochloride, is being used as a representative compound of (4,3-b)indoles.

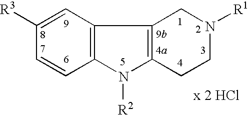

where R¹ and R³ are methyls, and

R² is 2-(6-methyl-3-pyridyl)-ethyl

This study is being performed essentially as described for Example 6 except that approximately 60 animals are randomized into six groups. At approximately 32 days, mice are given dimebon in the drinking water at the following concentrations:

Vehicle control (0 mg/kg/day)—3 groups

Dimebon 30 mg/kg/day—3 groups

At 3 different timepoints, following the initiation of dosing, animals are sacrificed, undergo perfusion/fixation, and have their brains and spinal cords isolated. At 6 weeks, 10 vehicle control and 10 dimebon animals are sacrificed. At 12 weeks, 10 vehicle control and 10 dimebon animals are sacrificed. At 18 weeks, 10 vehicle control and 10 dimebon animals are sacrificed. Just prior to perfusion/fixation at 10 a.m. in the morning on the day of sacrifice, plasma samples are obtained by direct cardiac puncture. Animals are evaluated by a blinded histopathologist, and motor neurons at the lumbar spinal level are manually quantitated. Analyses compare vehicle control animal neuron counts to dimebon animal neuron counts at each time point. Additional staining and histopathologic assessments may be performed to evaluate the mechanism of action of dimebon in this treatment model. Additional pharmacokinetic-pharmacodynamic analyses may be performed

### Example 10

### Evaluation of the Effect of Dimebon on Toxicity Induced by Ionomycin

The ability of dimebon to protect human glioblastoma cell lines from the neurotoxicant ionomycin was investigated. The neuroprotective effects of dimebon indicate that the compound has direct and broad neuroprotective properties on cell lines and would be expected to be beneficial in the treatment of ALS.

Two human neuroblastoma cell lines were used to perform these experiments: SK-N-SH cells and SY-SH5Y cells. SK-N-SH cells were maintained in EMEM supplemented with 10% FBS, at 37° C., 5% CO₂. SH-SY5Y cells were maintained in a 1:1 mixture of EMEM and F12 medium, supplemented with 10% FBS at 37° C., 5% CO₂.

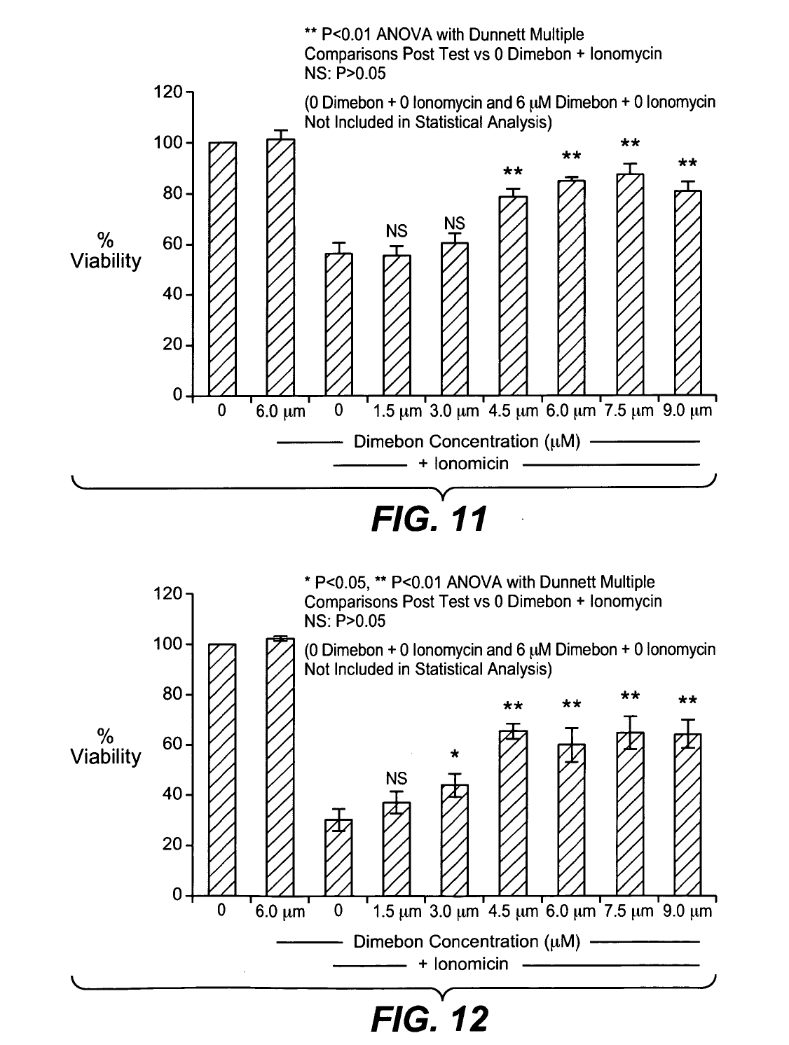

Cells were seeded at 3×10⁴ cells per well in 96-well plates containing 100 μl of the required medium. A day after seeding, cells were treated with different concentrations of ionomycin in MEM medium without serum (assay medium) in triplicate for 24 h a in final volume of 100 μl. Cell viability was determined by the MTS reduction assay as follows. MTS (20 μl) was added to each well for at least 1 h at 37° C. Absorbance at 490 nm was measured using a microplate reader. Dimebon at various concentrations was used to study the effect on ionomycin-treated cells. Cells were seeded at the same density as previously detailed. The cells were treated for 24 h with a solution containing 1.5 μM ionomycin and different concentrations of Dimebon in a final volume of 100 μl. Each experiment was performed in triplicate and the cell viability was determined by the MTS reduction assay. The results were graphed using control cells (incubated with assay medium only) as reference. Percent (%) Viability is the percent of MTS signal for each sample relative to the control (no Dimebon and no ionomycin treatment). Three independent experiments were considered for the statistical analysis. A non-parametric ANOVA followed by a Dunnett Multiple Comparisons Post Test analysis was used. FIGS. 11 and 12 illustrate the effect of Dimebon on Ionomycin-Induced Toxicity of SK-N-SH cells and SY-SH5Y cells, respectively.

### Example 11

### Evaluation of the Effect of Dimebon on Toxicity Induced by Serum Deprivation

The ability of dimebon to protect primary chick neurons from low serum was investigated. The neuroprotective effects of dimebon indicate that the compound has direct and broad neuroprotective properties and would be beneficial in the treatment of ALS.

Cells: Lohman Brown chicken embryo hybrids were used for the assay. One-day-old fertilised eggs were purchased from a local chicken breeder (Schropper Geflügel GmbH, Austria) and stored in the lab under appropriate conditions (12° C. and 80% humidity). At embryonic day 0 eggs were transferred into a breeding incubator and stored under permanent turning until embryonic day 8 at 37.8° C. and 55% humidity. Approximately five to six chicken embryos were used for isolation of neurons per experiment.

Eggs were wiped with 70% ethanol and cracked with large forceps at the blunt end. After decapitation of the embryo, the tissue covering the telencephalon was removed and hemispheres collected. After removing any loose tissue and remaining meningeal membranes, hemispheres were transferred into a dish containing nutrition medium. The tissue was dissociated mechanically by using a 1 ml pipette and by squeezing 3 times through a sterile nylon sieve with a pore size of 100 μm.

Poly-D-Lysine coated 96-well microtiter plates (Biocoat) were used to culture the cells. Culture medium (160 μl) containing 3×10⁵ cells/ml nutrition medium (48 000 cells/well) were added to each well of a microtiter plate Plates were kept at 37° C., 95% humidity and 5% CO₂ without change of media. Neurons begin to extend processes after a few hours in culture.

Low Serum Culture Conditions: The low serum medium used for the 2% growth factor withdrawal experiments described here includes EMEM with 1 g glucose/l and 2% FCS. The control medium includes DMEM with 4.5 g glucose/l and 5% Nu Serum. To prevent cell cultures from an infection with mycoplasm or other unwanted microorganism, gentamycin sulphate (0.1 mg/ml nutrition medium) was added to DMEM and EMEM.

Dimebon was applied to the cells on day 1 for the whole experimental period of 8 days. Viability of cells was determined with the MTT assay using a plate-reader (570 nM). This assay is based on the reduction of yellow MTT (3-(4,5-dimethylthiazol-2-yl)-2,5,diphenyl tetrazolium bromide), to dark blue formazan crystals by mitochondrial dehydrogenases (succinate dehydrogenase). Since this reaction is catalysed in living cells only the assay can be used for the quantification of cell viability. For the determination of cell viability, MTT solution was added to each well in a final concentration of 0.5 mg/ml. After 2 h the MTT containing medium was aspired. Cells were lysed with 3% SDS and formazan crystals were dissolved in Isopropanol/HCl. To estimate optical density a plate-reader (Anthos HT II) was used at wavelength 570 nM. Cell proliferation rate was expressed in optical density (OD).

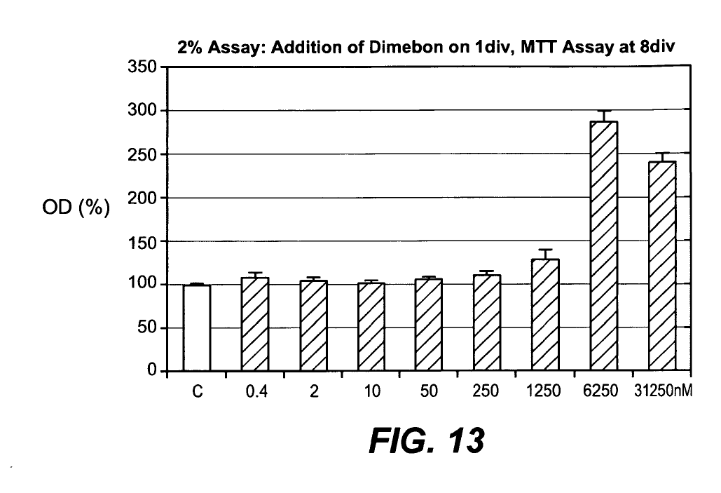

In the growth factor withdrawal assay Dimebon demonstrated a dose-dependent and statistically significant increase in OD570 nm in the MTT and AM-Calcein assays. Statistically significant differences compared to control were achieved at Dimebon concentrations of 1250 nM (p<0.05 for MTT and p<0.01 for AM-Calcein) and greater. A maximum effect in the MTT assay was achieved at a Dimebon concentration of 6250 nM which was approximately 287% above control. At the highest tested concentration (31250 nM) the effect in the MTT was less than what was achieved at a concentration of 6250 nM. Results are shown in FIG. 13.

### Example 12

### Use of Human Clinical Trials to Determine the Ability to Compounds of the Invention to Treat, Prevent and/or Delay the Onset and/or the Development of Amyotrophic Lateral Sclerosis

If desired, any of the hydrogenated pyrido[4,3-b]indoles (such as dimebon) or combination therapies described herein can also be tested in humans to determine the ability of the compound to treat, prevent and/or delay the onset and/or the development of ALS. Standard methods can be used for these clinical trials, such as those described in U.S. Pat. No. 5,527,814 or U.S. Pat. No. 5,780,489.

In one exemplary method, subjects with ALS are enrolled in a tolerability, pharmacokinetics and pharmacodynamics phase I study of a hydrogenated pyrido[4,3-b]indole using standard protocols such as those described in U.S. Pat. No. 5,780,489. Then a phase II, double-blind randomized controlled trial is performed to determine the efficacy of the hydrogenated pyrido[4,3-b]indole (see, for example, U.S. Pat. No. 5,780,489). The activity of the hydrogenated pyrido[4,3-b]indole can be compared to that of the anti-glutamate agent, Riluzole™, which is considered the “standard” treatment in clinical trials. Alternatively or additionally, the efficacy of a combination of the hydrogenated pyrido[4,3-b]indole and Riluzole™ can be compared to that of Riluzole™ alone. Subjects may be analyzed for the progression of ALS using the ALS functional rating score or analysis of specific ALS symptoms. Also, the length of survival can be compared between treatment groups (see, for example, U.S. Pat. No. 5,780,489).

### Example 13

### Use of Human Clinical Trials to Determine the Ability to Compounds of the Invention to Treat, Prevent and/or Delay the Onset and/or the Development of Amyotrophic Lateral Sclerosis

An exemplary clinical trial to determine the ability of any the hydrogenated pyrido[4,3-b]indoles (such as dimebon) or combination therapies described herein to treat, prevent and/or delay the onset and/or the development of ALS is described below. A phase 2, multi-center, randomized, double-blind, placebo-controlled trial is used. Approximately 100 subjects are enrolled in the trial at approximately 20 ALS treatment centers in the U.S. The trial includes a 9 month dosing period with a 3 week screening period and a 2 week safety follow-up period. The primary efficacy endpoint is the mean change in ALSFRS-R (ALS functional rating scale-revised). Secondary efficacy endpoints include tracheostomy-free survival, motor unit number estimation, and mean relative change in forced vital capacity. Safety, tolerability, and/or pharmacokinetics may also be measured. Regarding concomitant medications, riluzole, creatine, and co-enzyme Q are allowed provided that subjects are on a stable dose for at least 30 days prior to enrollment. Other experimental ALS disease-modifying therapies are excluded for 30 days prior to enrollment and during the study period. Potent inhibitors of CYP2D6 are excluded for 30 days prior to enrollment and during the study period.

A phase 3, multi-national, randomized, double-blind, placebo-controlled trial may also be performed. Approximately 450 subjects are enrolled at approximately 25 ALS treatment centers in the US and 20 treatment centers in Europe. The trial includes a 12-18 month dosing period (the duration of which depends on the phase 2 results), a 3 week screening period, and a 2 week safety follow-up period. The primary endpoint is tracheostomy-free survival. The secondary endpoints include mean change in ALSFRS-R, mean change in forced vital capacity, quality of life, and safety. Regarding concomitant medications, riluzole, creatine, and co-enzyme Q are allowed provided that subjects are on a stable dose for at least 30 days prior to enrollment. Other experimental ALS disease-modifying therapies are excluded for 30 days prior to enrollment and during the study period. Potent inhibitors of CYP2D6 are excluded for 30 days prior to enrollment and during the study period.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

All references, publications, patents, and patent applications disclosed herein are hereby incorporated by reference in their entirety.

1. A method of treating amyotrophic lateral sclerosis (ALS) in an individual in need thereof, the method comprising administering to an individual an effective amount of a hydrogenated pyrido(4,3-b)indole of the formula: 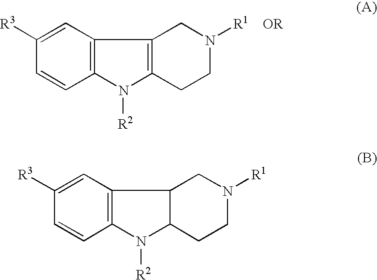
wherein:
R¹ is selected from a lower alkyl or aralkyl;
R² is selected from a hydrogen, aralkyl or substituted heteroaralkyl; and
R³ is selected from hydrogen, lower alkyl or halo,
or pharmaceutically acceptable salt thereof.

2-4. (canceled)

5. The method of claim 1, wherein aralkyl is PhCH₂— and substituted heteroaralkyl is 6-CH₃-3-Py-(CH₂)₂—.

6. The method of claim 1, wherein
R¹ is selected from CH₃—, CH₃CH₂—, or PhCH₂—
R² is selected from H—, PhCH₂—, or 6-CH₃-3-Py-(CH₂)₂—
R³ is selected from H—, CH₃— or Br—.

7. The method of claim 1, wherein the hydrogenated pyrido(4,3-b)indole is selected from the group consisting of:
cis(±) 2,8-dimethyl-2,3,4,4a,5,9b-hexahydro-1H-pyrido[4,3-b]indole;
2-ethyl-2,3,4,5-tetrahydro-1H-pyrido[4,3-b]indole;
2-benzyl-2,3,4,5-tetrahydro-1H-pyrido[4,3-b]indole;
2,8-dimethyl-5-benzyl-2,3,4,5-tetrahydro-1H-pyrido[4,3-b]indole;
2-methyl-5-(2-methyl-3-pyridyl)ethyl-2,3,4,5-tetrahydro-1H-pyrido[4,3-b]indole;
2,8-dimethyl-5-(2-(6-methyl-3-pyridyl)ethyl)-2,3,4,5-tetrahydro-1H-pyrido[4,3-b]indole;
2-methyl-2,3,4,5-tetrahydro-1H-pyrido[4,3-b]indole;
2,8-dimethyl-2,3,4,5-tetrahydro-1H-pyrido[4,3-b]indole;
2-methyl-8-bromo-2,3,4,5-tetrahydro-1H-pyrido[4,3-b]indole.

8. The method of claim 7, wherein the hydrogenated pyrido(4,3-b)indole is 2,8-dimethyl-5-(2-(6-methyl-3-pyridyl)ethyl)-2,3,4,5-tetrahydro-1H-pyrido[4,3-b]indole.

9. (canceled)

10. The method of claim 1, wherein the pharmaceutically acceptable salt is a hydrochloride acid salt.

11. The method of claim 1, wherein the hydrogenated pyrido(4,3-b)indole is 2,8-dimethyl-5-(2-(6-methyl-3-pyridyl)ethyl)-2,3,4,5-tetrahydro-1H-pyrido[4,3-b]indole dihydrochloride.

12-18. (canceled)

19. A method of slowing the progression of amyotrophic lateral sclerosis (ALS) in an individual who has a mutated or abnormal gene associated with ALS or who has been diagnosed with ALS, the method comprising administering to the individual an effective amount of a hydrogenated pyrido(4,3-b)indole of the formula: 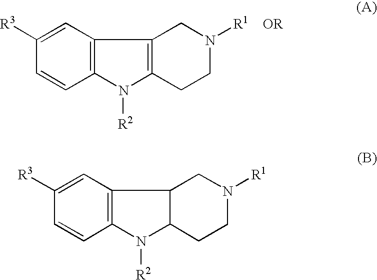
wherein:
R¹ is selected from a lower alkyl or aralkyl;
R² is selected from a hydrogen, aralkyl or substituted heteroaralkyl; and
R³ is selected from hydrogen, lower alkyl or halo,
or pharmaceutically acceptable salt thereof.

20-22. (canceled)

23. The method of claim 19, wherein aralkyl is PhCH₂— and substituted heteroaralkyl is 6-CH₃-3-Py-(CH₂)₂—.

24. The method of claim 19, wherein
R¹ is selected from CH₃—, CH₃CH₂—, or PhCH₂—
R² is selected from H—, PhCH₂—, or 6-CH₃-3-Py-(CH₂)₂—
R³ is selected from H—, CH₃— or Br—.

25. The method of claim 19, wherein the hydrogenated pyrido(4,3-b)indole is selected from the group consisting of:
cis(±) 2,8-dimethyl-2,3,4,4a,5,9b-hexahydro-1H-pyrido[4,3-b]indole;
2-ethyl-2,3,4,5-tetrahydro-1H-pyrido[4,3-b]indole;
2-benzyl-2,3,4,5-tetrahydro-1H-pyrido[4,3-b]indole;
2,8-dimethyl-5-benzyl-2,3,4,5-tetrahydro-1H-pyrido[4,3-b]indole;
2-methyl-5-(2-methyl-3-pyridyl)ethyl-2,3,4,5-tetrahydro-1H-pyrido[4,3-b]indole;
2,8-dimethyl-5-(2-(6-methyl-3-pyridyl)ethyl)-2,3,4,5-tetrahydro-1H-pyrido[4,3-b]indole;
2-methyl-2,3,4,5-tetrahydro-1H-pyrido[4,3-b]indole;
2,8-dimethyl-2,3,4,5-tetrahydro-1H-pyrido[4,3-b]indole;
2-methyl-8-bromo-2,3,4,5-tetrahydro-1H-pyrido[4,3-b]indole.

26. The method of claim 25, wherein the hydrogenated pyrido(4,3-b)indole is 2,8-dimethyl-5-(2-(6-methyl-3-pyridyl)ethyl)-2,3,4,5-tetrahydro-1H-pyrido[4,3-b]indole.

27. (canceled)

28. The method of claim 19, wherein the pharmaceutically acceptable salt is a hydrochloride acid salt.

29. The method of claim 19, wherein the hydrogenated pyrido(4,3-b)indole is 2,8-dimethyl-5-(2-(6-methyl-3-pyridyl)ethyl)-2,3,4,5-tetrahydro-1H-pyrido[4,3-b]indole dihydrochloride.

30-36. (canceled)

37. A method of preventing or delaying development of amyotrophic lateral sclerosis (ALS) in an individual who is at risk of developing ALS, the method comprising administering to an individual an effective amount of a hydrogenated pyrido(4,3-b)indole of the formula: 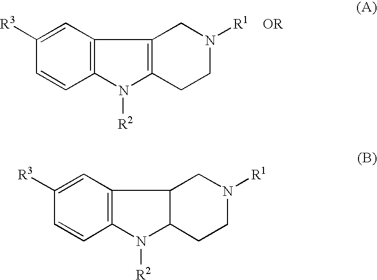
wherein:
R¹ is selected from a lower alkyl or aralkyl;
R² is selected from a hydrogen, aralkyl or substituted heteroaralkyl; and
R³ is selected from hydrogen, lower alkyl or halo,
or pharmaceutically acceptable salt thereof.

38-40. (canceled)

41. The method of claim 37, wherein aralkyl is PhCH₂— and substituted heteroaralkyl is 6-CH₃-3-Py-(CH₂)₂—.

42. The method of claim 37, wherein
R¹ is selected from CH₃—, CH₃CH₂—, or PhCH₂—
R² is selected from H—, PhCH₂—, or 6-CH₃-3-Py-(CH₂)₂—
R³ is selected from H—, CH₃— or Br—.

43. The method of claim 37, wherein the hydrogenated pyrido(4,3-b)indole is selected from the group consisting of:
cis(±) 2,8-dimethyl-2,3,4,4a,5,9b-hexahydro-1H-pyrido[4,3-b]indole;
2-ethyl-2,3,4,5-tetrahydro-1H-pyrido[4,3-b]indole;
2-benzyl-2,3,4,5-tetrahydro-1H-pyrido[4,3-b]indole;
2,8-dimethyl-5-benzyl-2,3,4,5-tetrahydro-1H-pyrido[4,3-b]indole;
2-methyl-5-(2-methyl-3-pyridyl)ethyl-2,3,4,5-tetrahydro-1H-pyrido[4,3-b]indole;
2,8-dimethyl-5-(2-(6-methyl-3-pyridyl)ethyl)-2,3,4,5-tetrahydro-1H-pyrido[4,3-b]indole;
2-methyl-2,3,4,5-tetrahydro-1H-pyrido[4,3-b]indole;
2,8-dimethyl-2,3,4,5-tetrahydro-1H-pyrido[4,3-b]indole;
2-methyl-8-bromo-2,3,4,5-tetrahydro-1H-pyrido[4,3-b]indole.

44. The method of claim 43, wherein the hydrogenated pyrido(4,3-b)indole is 2,8-dimethyl-5-(2-(6-methyl-3-pyridyl)ethyl)-2,3,4,5-tetrahydro-1H-pyrido[4,3-b]indole.

45. (canceled)

46. The method of claim 37, wherein the pharmaceutically acceptable salt is a hydrochloride acid salt.

47. The method of claim 37, wherein the hydrogenated pyrido(4,3-b)indole is 2,8-dimethyl-5-(2-(6-methyl-3-pyridyl)ethyl)-2,3,4,5-tetrahydro-1H-pyrido[4,3-b]indole dihydrochloride.

48-54. (canceled)

55. A kit comprising: (a) a hydrogenated pyrido(4,3-b)indole of the formula: 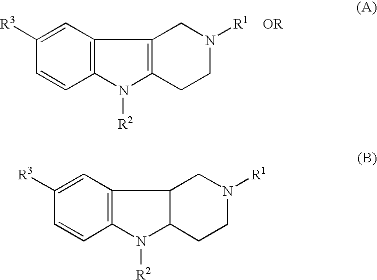
wherein:
R¹ is selected from a lower alkyl or aralkyl;
R² is selected from a hydrogen, aralkyl or substituted heteroaralkyl; and
R³ is selected from hydrogen, lower alkyl or halo,
or pharmaceutically acceptable salt thereof and (b) instructions for use of in the treatment, prevention, slowing the progression or delaying the onset and/or development of amyotrophic lateral sclerosis (ALS).

56-58. (canceled)

59. The kit of claim 55, wherein aralkyl is PhCH₂— and substituted heteroaralkyl is 6-CH₃-3-Py-(CH₂)₂—.

60. The kit of claim 55, wherein
R¹ is selected from CH₃—, CH₃CH₂—, or PhCH₂—
R² is selected from H—, PhCH₂—, or 6-CH₃-3-Py-(CH₂)₂—
R³ is selected from H—, CH₃— or Br—.

61. The kit of claim 55, wherein the hydrogenated pyrido(4,3-b)indole is selected from the group consisting of:
cis(±) 2,8-dimethyl-2,3,4,4a,5,9b-hexahydro-1H-pyrido[4,3-b]indole;
2-ethyl-2,3,4,5-tetrahydro-1H-pyrido[4,3-b]indole;
2-benzyl-2,3,4,5-tetrahydro-1H-pyrido[4,3-b]indole;
2,8-dimethyl-5-benzyl-2,3,4,5-tetrahydro-1H-pyrido[4,3-b]indole;
2-methyl-5-(2-methyl-3-pyridyl)ethyl-2,3,4,5-tetrahydro-1H-pyrido[4,3-b]indole;
2,8-dimethyl-5-(2-(6-methyl-3-pyridyl)ethyl)-2,3,4,5-tetrahydro-1H-pyrido[4,3-b]indole;
2-methyl-2,3,4,5-tetrahydro-1H-pyrido[4,3-b]indole;
2,8-dimethyl-2,3,4,5-tetrahydro-1H-pyrido[4,3-b]indole;
2-methyl-8-bromo-2,3,4,5-tetrahydro-1H-pyrido[4,3-b]indole.

62. The kit of claim 61, wherein the hydrogenated pyrido(4,3-b)indole is 2,8-dimethyl-5-(2-(6-methyl-3-pyridyl)ethyl)-2,3,4,5-tetrahydro-1H-pyrido[4,3-b]indole.

63. (canceled)

64. The kit of claim 55, wherein the pharmaceutically acceptable salt is a hydrochloride acid salt.

65. The kit of claim 55, wherein the hydrogenated pyrido(4,3-b)indole is 2,8-dimethyl-5-(2-(6-methyl-3-pyridyl)ethyl)-2,3,4,5-tetrahydro-1H-pyrido[4,3-b]indole dihydrochloride.

66-72. (canceled)

73. The method of any one of claim 1, 19, or 37, further comprising administering to the individual another compound or pharmaceutically acceptable salt thereof that is useful for treating, preventing and/or delaying the onset and/or development of ALS.

74. The kit of claim 55, further comprising another compound or pharmaceutically acceptable salt thereof that is useful for treating, preventing and/or delaying the onset and/or development of ALS.

75. A unit dosage form comprising (a) first therapy comprising a hydrogenated pyrido(4,3-b)indole of the formula: 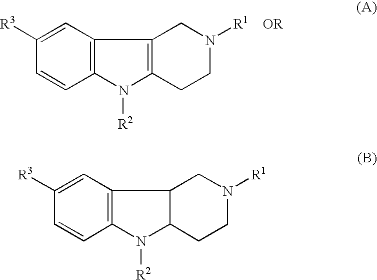
wherein:
R¹ is selected from a lower alkyl or aralkyl;
R² is selected from a hydrogen, aralkyl or substituted heteroaralkyl; and
R³ is selected from hydrogen, lower alkyl or halo,
or pharmaceutically acceptable salt thereof, (b) a second therapy comprising another compound or pharmaceutically acceptable salt thereof that is useful for treating, preventing and/or delaying the onset and/or development of ALS and (c) a pharmaceutically acceptable carrier.
